(19) United States
(12) Patent Application Publication
(10) Pub. No.: US 2024/0270847 A1
(43) Pub. Date: Aug. 15, 2024

(54) ANTIBODY BINDING CTLA-4 AND USE THEREOF

(71) Applicant: HARBOUR BIOMED (SHANGHAI) CO., LTD., Shanghai (CN)

(72) Inventors: Xin GAN, Shanghai (CN); Jinfeng XIANG, Shanghai (CN); Xiaolu TAO, Shanghai (CN); Jinfeng JI, Shanghai (CN); Xiaoxiang CHEN, Shanghai (CN)

(73) Assignee: HARBOUR BIOMED (SHANGHAI) CO., LTD., Shanghai (CN)

(21) Appl. No.: 18/287,822

(22) PCT Filed: Apr. 20, 2022
(86) PCT No.: PCT/CN2022/087922

§ 371 Date: Oct. 20, 2023

(30) Foreign Application Priority Data
Apr. 21, 2021 (CN) 202110429291.6
Apr. 14, 2022 (CN) 202210392059.4

(51) Int. Cl. C07K 16/28; A61K 9/00; A61K 39/00; A61P 35/00

(52) U.S. Cl. CPC C07K 16/2818; A61K 9/0019; A61P 35/00; A61K 2039/507; A61K 2039/545; C07K 2317/565; C07K 2317/622

(57) ABSTRACT

Provided are a method for depleting regulatory T cells (Tregs) and/or increasing the proportion of CD8+ T cells/Tregs in a subject, a method for treating a refractory cancer in a subject, a combination therapy for treating a cancer, a method for reducing side effects of treating a cancer using a CTLA4 antibody, a method for implementing long-term administration or administration when necessary for a cancer patient, an administration method for an anti-CTLA-4 single heavy chain antibody, a unit dose composition, the use of an anti-CTLA-4 single heavy chain antibody in preparation of a drug, a method for determining the administration dose of an anti-tumor drug, and a method for determining the administration form of an anti-tumor drug.

The present international patent application claims the benefits of Chinese Patent Application No. CN202110429291.6 filed on Apr. 21, 2021 and Chinese Patent Application No. 202210392059.4 filed on Apr. 14, 2022, which are incorporated herein by reference in their entirety.

### TECHNICAL FIELD

The present invention relates to the field of biomedicine, and in particular to a CTLA-4-binding antibody and use thereof in the treatment of cancer. More specifically, the present invention relates to a method for removing a regulatory T cell (Treg) and/or increasing the CD8+ T cell/Treg cell ratio in a subject, a method for treating refractory cancer in a subject, a combination therapy for treating cancer, a method for reducing side effects of a CTLA4 antibody in treating cancer, a method for achieving long-cycle administration or pro re nata (PRN) administration to a cancer patient, a method for administering anti-CTLA-4 single heavy-chain antibodies, a unit dose composition, use of anti-CTLA-4 single heavy-chain antibodies in preparing a medicament, a method for determining the dose of an anti-tumor drug, and a method for determining the form of administration of an anti-tumor drug.

### BACKGROUND

Cancer immunotherapy is a recent breakthrough in cancer treatment, which utilizes the patient's own immune system to attack tumor cells. Promotion of a strong CD8 T cell-dependent cytotoxic response in a tumor microenvironment is important for generating an effective anti-tumor immune response. However, tumors tend to evade immune surveillance by utilizing T cell inhibition mechanisms.

Activation of a T cell requires stimulation with double-positive signals, i.e., a first signal and a second signal. The first signal is achieved by the antigen recognition process, i.e. specific binding of TCR to a MHC molecule-antigen peptide complex. The second signal is activated by means of synergistic costimulatory molecules, wherein the CD28 molecule on the surface of a T cell binds to the corresponding ligand CD80/CD86 (B7-1/B7-2) on the surface of an antigen-presenting cell, eventually initiating an immune response. CTLA-4 is an important immune checkpoint that negatively regulates T cell activation/proliferation. Activated T cells tend to achieve their negative regulation by up-regulating the expression of CTLA-4 and by competitively binding to the ligand CD80/CD86 (B7-1/B7-2) with CD28, preventing the activation and proliferation of T cells. Tumor cells also often inhibit the immune response by expressing CTLA-4, thereby achieving “immune escape”. Therefore, the CTLA-4 inhibitor drug developed on the basis of the theory described above promotes the activation of immune cells by blocking CTLA-4-related inhibition signals to achieve the recovery and enhancement of anti-tumor immune functions.

CTLA-4 has been shown to have excellent anti-tumor effects in a variety of tumors as a single drug and in combination therapy. For example, ipilimumab that has been approved shows good efficacy in treating tumors such as advanced metastatic melanoma. In addition, clinical trials from different anti-CTLA-4 drugs all show nearly doubled efficacy gains in the anti-CTLA-4 high dose group compared to the low dose group; at the same time, high dose single drug groups also show good clinical benefits. The combination therapy also shows significant efficacy gains in several types of advanced solid tumors: by using CTLA-4 in combination, the combination treatment group exhibits a 2-3-fold increase in ORR compared to the original monotherapy: after CTLA-4 is combined in the treatment of part of refractory tumor species or tumor species insensitive to single immunotherapy (such as sarcoma, stomach cancer, cervical cancer, and the like), the efficacy shows a breakthrough¹⁻²³.

Despite significant efficacy gains, current anti-CTLA-4 monoclonal antibodies and combination therapies are limited by their toxic characteristics, and thus the potential for further improvement of their efficacy is limited. Currently limited by toxic ceilings, the clinical application mode of the target is generally a low dose non-maintenance therapy. However, the existing data show that for tumor species such as HCC, the efficacy is significantly positively correlated with the dose of CTLA-4, and even the currently approved administration form (ipilimumab 4 cycles+nivolumab maintenance) still has the practical problem that most patients are not easy to be tolerable, causing discontinuation, so that the necessity and great value of developing a next-generation product for improving the safety of the target drug are highlighted. Therefore, further exploring of next-generation antibodies will potentially bring higher clinical benefits through enhanced efficacy and improved safety.

### SUMMARY

In one aspect, the present disclosure relates to an anti-CTLA-4 single heavy-chain antibody, such as a fully human single heavy-chain antibody. Compared with a traditional antibody with a classical antibody structure (namely, an antibody structure comprising two heavy chains and two light chains), the anti-CTLA-4 single heavy-chain antibody of the present disclosure has the characteristics of lower steric hindrance, high affinity, high tissue permeability, high stability and the like. The inventors have surprisingly found that the anti-CTLA-4 single heavy-chain antibody of the present disclosure achieves low exposure in vivo and adverse effects that have a narrow range and are controllable compared to conventional antibodies: it has a relatively short half-life but long duration of efficacy (including anti-tumor effects and Treg removing effects).

In one aspect, the present disclosure relates to a method for removing a regulatory T cell (Treg) and/or increasing the CD8+ T cell/Treg cell ratio in a subject. According to an embodiment of the present invention, the method comprises administering to the subject an anti-CTLA-4 single heavy-chain antibody. For example, a fully human single heavy-chain antibody. In some embodiments, the subject is a cancer patient. In some embodiments, the subject is a patient with endometrial cancer, non-clear cell renal cell carcinoma, clear cell renal cell carcinoma, non-small cell lung cancer, head and neck cancer, breast cancer, castration-resistant prostate cancer, testicular cancer, urothelial cancer, liver cancer, esophageal cancer, mesothelioma, melanoma, or neuroendocrine neoplasm.

In one aspect, the present disclosure relates to a method for treating refractory cancer in a subject. According to an embodiment of the present invention, the method comprises administering to the subject an anti-CTLA-4 single heavy-chain antibody. In some embodiments, the refractory cancer is not responsive or tolerable to one or more immune checkpoint inhibitors such as a PD-1/PD-L1 axis signaling pathway inhibitor. In some embodiments, the cancer is HCC. In other embodiments, the cancer is castration-resistant prostate cancer (CRPC).

In one aspect, the present disclosure relates to a method for treating cancer. According to an embodiment of the present invention, the method comprises the step of administering to the subject an anti-CTLA-4 single heavy-chain antibody and a second therapeutic agent. In some embodiments, the second therapeutic agent is selected from one or more immune checkpoint inhibitors, such as a PD-1/PD-L1 axis signaling pathway inhibitor.

In some embodiments, the second therapeutic agent is an anti-PD-1 monoclonal antibody, such as perbrolizumab, nivolumab, or toripalimab.

In some embodiments, the second therapeutic agent is an antibody or an antigen-binding fragment that is targeted to HER-2, HER-3, EGFR, EpCAM, PD-1/PD-L1, CD27, CD28, ICOS, CD40, CD122, OX43, 4-1BB, GITR, B7-H3, B7-H4, BTLA, LAG-3, CD15, CD52, CA-125, CD34, A2AR, VISTA, TIM-3, KIR, CD30, CD33, CD38, CD20, CD24, CD90, CA-15-3, CA-19-9, CEA, CD99, CD117, CD31, CD44, CD123, CD133, ABCB5, CD45, or the like.

In some embodiments, the second therapeutic agent is a chemotherapeutic agent. For example, the second therapeutic agent is selected from paclitaxel, cisplatin, carboplatin, gemcitabine, pemetrexed, oxaliplatin, epirubicin, fluorouracil, and the like.

In some embodiments, the second therapeutic agent is administered before the anti-CTLA-4 single heavy-chain antibody, simultaneously with the anti-CTLA-4 single heavy-chain antibody, or after the anti-CTLA-4 single heavy-chain antibody.

In one aspect, the present disclosure relates to a method for preventing and/or reducing side effects of a CTLA4 antibody in treating cancer. According to an embodiment of the present invention, the method comprises administering to the subject an anti-CTLA-4 single heavy-chain antibody.

In one aspect, the present disclosure relates to a method for administering an anti-CTLA-4 single heavy-chain antibody. According to an embodiment of the present invention, the method comprises administering the anti-CTLA-4 single heavy-chain antibody to a patient by intravenous drip infusion, the anti-CTLA-4 single heavy-chain antibody is provided in the form of a 0.9% sodium chloride or 5% glucose solution, the concentration of the anti-CTLA-4 single heavy-chain antibody in the solution is 0.1 mg/mL to 10.0 mg/mL, and the time for the intravenous drip infusion is no more than 4 hours.

In some embodiments, the method described above comprises administering to the subject the anti-CTLA-4 single heavy-chain antibody at a dose of 0.2 mg to 1 mg/kg body weight.

In some embodiments, the administration cycle included in the method described above is once every week to every 12 weeks. For example, the administration cycle is once every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks. It should be noted that the “administration cycle” described herein refers to the time between two adjacent administrations.

In some embodiments, the anti-CTLA-4 single heavy-chain antibody is administered to the subject at a dose of 0.3-0.6 mg/kg, 0.3-0.55 mg/kg, 0.3-0.5 mg/kg, 0.3-0.45 mg/kg, 0.35-0.6 mg/kg, 0.35-0.55 mg/kg, 0.35-0.5 mg/kg, 0.35-0.45 mg/kg, 0.45-0.6 mg/kg, 0.45-0.55 mg/kg, or 0.45-0.5 mg/kg.

In some embodiments, the anti-CTLA-4 single heavy-chain antibody is administered to the subject at a dose of 0.3-0.6 mg/kg, 0.3-0.55 mg/kg, 0.3-0.5 mg/kg, 0.3-0.45 mg/kg, 0.35-0.6 mg/kg, 0.35-0.55 mg/kg, 0.35-0.5 mg/kg, 0.35-0.45 mg/kg, 0.45-0.6 mg/kg, 0.45-0.55 mg/kg, or 0.45-0.5 mg/kg once every 3 weeks.

In some embodiments, the anti-CTLA-4 single heavy-chain antibody is administered to the subject at a dose of 0.3 mg/kg once every week.

In some embodiments, the anti-CTLA-4 single heavy-chain antibody is administered to the subject at a dose of 0.45 mg/kg once every 3 weeks.

In some embodiments, the anti-CTLA-4 single heavy-chain antibody is administered to the subject at a dose of 0.6 mg/kg once every 3 weeks.

In one aspect, the present disclosure relates to a method for achieving long-cycle administration or PRN administration to a cancer patient. According to an embodiment of the present invention, the method comprises administering to the subject an anti-CTLA-4 single heavy-chain antibody.

In some embodiments, the long-cycle administration means that the administration cycle is not less than 4 weeks, preferably not less than 6 weeks, 7 weeks, 8 weeks, 9 weeks, 10 weeks, 11 weeks, or 12 weeks, such as 6 weeks or 12 weeks.

In some embodiments, the subject's inability to maintain stable disease (SD), partial response (PR), or complete response (CR) state is indicative of PRN administration. SD means that the sum of the maximum diameters of the target lesions is reduced and does not reach PR, or increased and does not reach PD; PR means that the sum of the maximum diameters of the target lesions is reduced by more than or equal to 30%, which is maintained for at least 4 weeks; CR means that all target lesions disappear, no new lesions appear, and the tumor marker is normal, which are maintained for at least 4 weeks; PD means that the sum of the maximum diameters of the target lesions is increased by at least 20%, or a new lesion appears.

In some embodiments, the subject is unable to maintain SD, PR, or CR state, and the anti-CTLA-4 single heavy-chain antibody is administered to the subject until the subject recovers the SD, PR, or CR state.

In some embodiments, the anti-CTLA-4 single heavy-chain antibody is administered for a duration of 2-4 administration cycles.

In some embodiments, the anti-CTLA-4 single heavy-chain antibody is administered to the subject at a dose of 0.2 mg to 1 mg/kg body weight, preferably 0.3-0.6 mg/kg.

In one aspect, the present disclosure relates to a unit dose composition. According to an embodiment of the present invention, 20-200 mg of an anti-CTLA-4 single heavy-chain antibody is included. For example, 30-200 mg, 40-200 mg, 50-200 mg, 60-200 mg, 70-200 mg, 80-200 mg, 90-200 mg, 100-200 mg, 60-100 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, and 200 mg.

In one aspect, the present disclosure relates to use of an anti-CTLA-4 single heavy-chain antibody in preparing a medicament for treating a CTLA-4-related disease such as cancer in a subject, wherein the medicament is formulated into a form administered to a patient at a dose of 0.2 mg to 1 mg/kg.

In some embodiments of the method, the unit dose composition, or the use described above, the anti-CTLA-4 single heavy-chain antibody has a CDR, wherein the CDR has an amino acid sequence set forth in any one of SEQ ID NOs: 1-24.

In some embodiments of the method, the unit dose composition, or the use described above, the anti-CTLA-4 single heavy-chain antibody has CDR1, CDR2, and CDR3, wherein the CDR1 has an amino acid sequence set forth in any one of SEQ ID NOs: 1-8: the CDR2 has an amino acid sequence set forth in any one of SEQ ID NOs: 9-16: the CDR3 has an amino acid sequence set forth in any one of SEQ ID NOs: 17-24. In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 1, 9, and 17, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 2, 10, and 18, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 3, 11, and 19, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 4, 12, and 20, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 5, 13, and 21, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 6, 14, and 22, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 7, 15, and 23, respectively.

In some embodiments of the method, the unit dose composition, or the use described above, the anti-CTLA-4 single heavy-chain antibody has CDR1, CDR2, and CDR3, wherein the CDR1, the CDR2, and the CDR3 have amino acid sequences set forth in SEQ ID NOs: 8, 16, and 24, respectively.

In some embodiments of the method, the unit dose composition, or the use described above, the anti-CTLA-4 single heavy-chain antibody has a heavy chain variable region, wherein the heavy chain variable region has an amino acid sequence set forth in any one of SEQ ID NOs: 25-32.

In some embodiments of the method, the unit dose composition, or the use described above, the anti-CTLA-4 single heavy-chain antibody has an amino acid sequence set forth in any one of SEQ ID NOs: 33-40.

In one aspect, the present disclosure relates to a method for administering an anti-CTLA-4 single heavy-chain antibody, wherein the anti-CTLA-4 single heavy-chain antibody has an amino acid sequence set forth in SEQ ID NO. 33. According to an embodiment of the present invention, the method comprises administering the anti-CTLA-4 single heavy-chain antibody to a patient by intravenous drip infusion, the anti-CTLA-4 single heavy-chain antibody is provided in the form of a 0.9% sodium chloride or 5% glucose solution, the concentration of the anti-CTLA-4 single heavy-chain antibody in the solution is 0.1 mg/mL to 10.0 mg/mL, and the time for the intravenous drip infusion is no more than 4 hours.

In some embodiments, the anti-CTLA-4 single heavy-chain antibody is administered to the patient at a dose of 0.2 mg to 1 mg/kg, preferably at a dose of 0.3 mg to 0.6 mg/kg. In some embodiments, the administration cycle is once every week to every 12 weeks. In some embodiments, the administration cycle is once every week, every 2 weeks, every 3 weeks, every 4 weeks, every 5 weeks, every 6 weeks, every 7 weeks, every 8 weeks, every 9 weeks, every 10 weeks, every 11 weeks, or every 12 weeks.

In some embodiments, the anti-CTLA-4 single heavy-chain antibody is administered to the patient at a dose of 0.3-0.6 mg/kg, 0.3-0.55 mg/kg, 0.3-0.5 mg/kg, 0.3-0.45 mg/kg, 0.35-0.6 mg/kg, 0.35-0.55 mg/kg, 0.35-0.5 mg/kg, 0.35-0.45 mg/kg, 0.45-0.6 mg/kg, 0.45-0.55 mg/kg, or 0.45-0.5 mg/kg once every 3 weeks.

In some embodiments, the anti-CTLA-4 single heavy-chain antibody is administered to the patient at a dose of 0.3 mg/kg once every week.

In some embodiments, the anti-CTLA-4 single heavy-chain antibody is administered to the patient at a dose of 0.45 mg/kg once every 3 weeks.

In some embodiments, the anti-CTLA-4 single heavy-chain antibody is administered to the patient at a dose of 0.6 mg/kg once every 3 weeks.

In one aspect, the present disclosure relates to a unit dose composition. According to an embodiment of the present invention, the unit dose composition comprises 60-100 mg of the anti-CTLA-4 single heavy-chain antibody, wherein the anti-CTLA-4 single heavy-chain antibody has the amino acid sequence described above. For example, the anti-CTLA-4 single heavy-chain antibody is selected from an amino acid sequence set forth in any one of SEQ ID NOs: 33-40. It should be noted that the “unit dose composition” described herein refers to a dose composition suitable for single administration, i.e., the unit dose composition contains an amount of an active ingredient suitable for single administration. The unit dose composition according to an embodiment of the present invention has high safety and good efficacy in single administration. In some embodiments, the unit dose composition comprises 80 mg of an anti-CTLA-4 single heavy-chain antibody.

In one aspect, the present disclosure relates to use of an anti-CTLA-4 single heavy-chain antibody in preparing a medicament for treating a CTLA-4-related disease such as cancer, wherein the anti-CTLA-4 single heavy-chain antibody has the amino acid sequence described above. For example, the anti-CTLA-4 single heavy-chain antibody is selected from an amino acid sequence set forth in any one of SEQ ID NOs: 33-40. The medicament is formulated into a form administered to a patient at a dose of 0.2 mg to 1 mg/kg.

In some embodiments, the medicament is formulated into a form administered at a dose of 0.3 mg/kg to 0.6 mg/kg once every 3 weeks; preferably, the medicament is formulated into a form administered at a dose of 0.3-0.45 mg/kg once every 3 weeks; preferably, the medicament is formulated into a form administered at a dose of 0.45 mg/kg once every 3 weeks.

In one aspect, the present disclosure relates to a “Tick-Tock pendulum movement” type method for determining the administration dose of an anti-tumor drug. According to an embodiment of the present invention, the method comprises: 1) performing a dose escalation experiment on a test drug in a wide range of tumor species according to a preset dose gradient so as to obtain data on efficacy and toxicity of the test drug under a preset dose: 2) determining, on the basis of the data on efficacy and toxicity of the test drug under a preset dose, whether the test drug is subjected to a dose expansion experiment in a specific tumor species under the preset dose: 3) repeating the step 2) in order to determine whether the test drug is subjected to a dose expansion experiment in a specific tumor species under a higher preset dose.

In some embodiments, the dose escalation experiment and the dose expansion experiment can be performed simultaneously: namely, in the process of dose escalation, once a dose group with reliable safety and efficacy signal generation occurs, the dose expansion of the dose in a specific tumor species is performed, meanwhile, the escalation of higher doses in extensive solid tumors is continued, and once the next “expandable dose” is determined, the expansion is continued according to the method described above. Finally, data of different dose levels in the same tumor species in the “expansion” part and data of the same dose level in specific tumor species are obtained, and finally, the “optimum dose for various tumor species” is selected to enter the subsequent phase II and phase III (PH 2/3) development.

In some embodiments, the method comprises “dose escalation” and “dose expansion”; the “dose escalation” part is as follows: The decision of “upgrade”, “stay”, or “downgrade” is performed according to the i3+3 dose escalation algorithm (FIGS. 1 and 2) and according to the combination of the dose limiting toxicity and the comprehensive toxicity performance at each dose level with the algorithm, and the dose group in which a “efficacy signal” is obtained is determined. The steps of the “dose expansion” are: According to the PD/PK characteristics and the efficacy profile exhibited in each dose group, evaluation is performed, and after regular scientific review and discussion to determine whether the dose “has a good benefit/risk ratio”, dose expansion in a specific tumor species can be initiated from a certain dose. In some embodiments, dose escalation is continued to further explore the safety margin of the drug and further determine the next dose with a good benefit/risk ratio; if the “benefit/risk ratio” of a higher dose group is still deemed to be acceptable and have the potential to expand by the scientific review described above, the expansion in a specific tumor species will likewise be performed in the manner described above.

In some embodiments, the anti-tumor drug is an anti-CTLA-4 single heavy-chain antibody.

In some embodiments, the CDRs, the heavy chain variable region, and the heavy chain of the anti-CTLA-4 single heavy-chain antibody have amino acid sequences set forth in SEQ ID NOs: 1-40.

In one aspect, the present disclosure relates to a method for determining the form of administration of an anti-tumor drug. According to an embodiment of the present invention, the method comprises administering a test drug to a test model, and performing a test at a preset administration dose, number of administrations, and administration cycle so as to obtain a dominant administration form of the test drug, wherein that SD, PR, or CR of the test model is in a stable state is an indication of the dominant administration form.

In some embodiments, the method further comprises administering a second therapeutic agent to the test model, and performing a test at a preset administration dose, number of administrations, and administration cycle so as to obtain a dominant administration form of the second therapeutic agent, wherein that SD, PR, or CR of the test model is still in a stable state is an indication of the dominant administration form of the second therapeutic agent.

In some embodiments, SD, PR, or CR of the test model cannot maintain a stable state, and the test drug is administered to the test model again so that SD, PR, or CR of the test model is in a stable state again.

In some embodiments, in addition to the preset administration duration and administration dose or administration cycle of the test anti-tumor drug, it is allowed to increase an extra administration cycle, and to add a new queue to explore a new administration interval. The possible outcomes of the exploration experiment are: It is possible that a specific tumor species has a specific administration dose and interval pattern and number of cycles: when necessary, the “pro re nata” (PRN) administration pattern is further explored, that is, when the preset administration duration is over (such as 4 administration cycles), if the result is SD or PR and CR, the second drug (such as PD-1) is used to maintain, regular tumor assessment is performed during the subsequent treatment process, and if disease progression or progression trend is possible, the test anti-tumor drug such as an anti-CTLA-4 single heavy-chain antibody is temporarily added for several administration cycles (such as 2-4 administration cycles) for “consolidation” treatment purpose. Until the patient has stable disease or response, the logic described above is discontinued, or disease progression occurs, and the present drug regimen is abandoned.

In some embodiments, the CDRs, the heavy chain variable region, and the heavy chain of the anti-CTLA-4 single heavy-chain antibody have amino acid sequences set forth in SEQ ID NOs: 1-40.

In one aspect, the present disclosure relates to an anti-CTLA-4 single heavy-chain antibody for removing a regulatory T cell (Treg) and/or increasing the CD8+ T cell/Treg cell ratio in a subject.

In one aspect, the present disclosure relates to an anti-CTLA-4 single heavy-chain antibody for treating refractory cancer in a subject.

In one aspect, the present disclosure relates to an anti-CTLA-4 single heavy-chain antibody and a second therapeutic agent for treating cancer.

In one aspect, the present disclosure relates to an anti-CTLA-4 single heavy-chain antibody for reducing side effects of a CTLA4 antibody in treating cancer.

In one aspect, the present disclosure relates to an anti-CTLA-4 single heavy-chain antibody for achieving long-cycle administration or PRN administration to a cancer patient.

The additional technical features and technical effects of the technical solutions described above are the same as those described above, which will not be repeated here.

### BRIEF DESCRIPTION OF THE DRAWINGS

FIG. 1 is a schematic diagram of “dose escalation” and “dose expansion” experiments according to an embodiment of the present invention.

FIG. 2 is a diagram showing an i3+3 dose escalation algorithm according to an embodiment of the present invention.

FIG. 3 is a diagram showing the results of killing of Treg cells by the anti-CTLA-4 single heavy-chain antibody according to an embodiment of the present invention: ISO: isotype control.

FIG. 4 is a diagram showing the results of killing of T_{reg} cells in CD45⁺ cells in a tumor sample by the anti-CTLA-4 single heavy-chain antibody according to an embodiment of the present invention: G1: hIgG1, 10 mg/kg: G2: an ipilimumab mimic, 10 mg/kg; G3: hIgG1 HCAb, 5.4 mg/kg: G4: HBM4003, 5.4 mg/kg: G5: HBM4003, 1.5 mg/kg.

FIG. 5 is a diagram showing mean serum concentration-time curves of the anti-CTLA-4 single heavy-chain antibody after intravenous administration at doses of 1 mg/kg and 5 mg/kg according to an embodiment of the present invention.

FIG. 6 is a diagram showing mean serum concentration-time curves of the anti-CTLA-4 single heavy-chain antibody after intravenous administration at single doses of 1, 3, and 10 mg/kg in cynomolgus monkeys according to an embodiment of the present invention.

FIG. 7 shows pharmacokinetic results for the anti-CTLA-4 single heavy-chain antibody in a patient according to an embodiment of the present invention.

A and B: anti-CTLA-4 single heavy-chain antibody concentration in serum-time curves of CID1-CID7 after the first administration at 0.3 mg/kg and CID1-C1D21 after the first administration at 0.45 mg/kg and 0.6 mg/kg, wherein A is plotted in logarithmic scale, and B is plotted in linear scale. C and D: anti-CTLA-4 single heavy-chain antibody concentration in serum-time curves of CID22-C1D28 after multiple administrations at 0.3 mg/kg, wherein C is plotted in logarithmic scale, and D is plotted in linear scale.

### DETAILED DESCRIPTION OF THE INVENTION

Unless otherwise defined herein, scientific and technical terms and their abbreviations used in connection with the present invention shall have the meaning commonly understood by those of ordinary skill in the art to which the present invention belongs. Some of the terms and abbreviations used herein are listed below.
Antibody: Ab; immunoglobulin: Ig;
heavy chain, HC; light chain, LC;
heavy chain variable region, VH;
heavy chain constant region, CH;
light chain variable region, VL;
light chain constant region, CL;
complementary determining region, CDR;
Fab fragment: antigen-binding fragment, Fab;
Fc region: fragment crystallizable region, Fc;
monoclonal antibody, mAb;
single heavy-chain antibody: antibody lacking light chains

The term “antibody” as referred to in the present disclosure is a single heavy-chain antibody. The antibody also includes a murine antibody, a humanized antibody, a chimeric antibody, a human antibody, and antibodies of other sources. The antibody may contain additional alterations, such as non-natural amino acids, mutations in Fc effector function, and mutations in glycosylation sites. The antibody also includes post-translationally modified antibodies, fusion proteins comprising the antigenic determinants of the antibody, and immunoglobulin molecules containing any other modifications to the antigen-recognition site, so long as these antibodies exhibit the desired bioactivity. In other words, the antibody includes an immunoglobulin molecule and an immunologically active fragment of an immunoglobulin molecule, that is, a molecule that contains at least one antigen-binding domain.

As used herein, “variable region” (heavy chain variable region VH and light chain variable region VL) refers to paired light and heavy chain domain moieties that are directly involved in binding of an antibody to an antigen. Each VH and VL region consists of three hypervariable regions or complementary determining regions (CDRs) and four framework regions (FRs) arranged from N-terminus to C-terminus in the following order: FR1, CDR1, FR2, CDR2, FR3, CDR3, FR4.

As used herein, the term “CDR” refers to the complementary determining regions within an antibody variable sequence. For each variable region, there are three CDRs in each variable region of the heavy and light chains, which are referred to as CDR1, CDR2, and CDR3. The exact boundaries of these CDRs are defined differently according to different systems. The system described by Kabat et al. (Kabat et al., Sequences of Proteins of Immunological Interest (National Institutes of Health, Bethesda, Md. (1987) and (1991)) provides not only a clear residue numbering system applicable to antibody variable regions, but also residue boundaries defining the three CDRs. Those CDRs may be referred to as Kabat CDRs. Each complementary determining region may comprise amino acid residues of a “complementary determining region” as defined by Kabat. Chothia et al. (Chothia & Lesk, J. mol. Biol, 196: 901-917 (1987) and Chothia et al., Nature 342:877-883 (1989)) have found that certain sub-portions within the Kabat CDRs adopt almost identical peptide backbone conformation, although with diversity at the amino acid sequence level. Those sub-portions are referred to as L1, L2, and L3, or H1, H2, and H3, respectively, where “L” and “H” represent the light and heavy chain regions, respectively. Those regions may be referred to as Chothia CDRs, which have boundaries that overlap with those of Kabat CDRs. There are some other CDRs whose boundaries may not be defined strictly following one of the above systems, but will still overlap with those of the Kabat CDRs. CDRs defined according to any of these systems may be used in the methods used herein, although CDRs defined by Kabat or Chothia are used in preferred embodiments. The present application uses the Kabat system to define CDR sequences.

In some embodiments, the amino acid modification does not alter the CDR sequences of the antibody, that is, the amino acid modification is made in the framework region (FR) of the variable region.

In some embodiments, the one or several amino acid modifications refer to 1-10 amino acid modifications or 1-5 amino acid modifications, such as 1, 2, 3, 4, 5, 6, 7, 8, 9, or amino acid modifications.

In some embodiments, the amino acid modification is selected from substitution, deletion, addition, and/or insertion of an amino acid residue. In some embodiments, the amino acid modification is an amino acid substitution, such as a conservative substitution.

In some embodiments of the single heavy-chain antibody of the present disclosure, the antibody has the following VH:
a. a VH comprising an amino acid sequence having at least 80% sequence identity, such as at least 90%, at least 95%, at least 98%, or at least 99% sequence identity, to an amino acid sequence of SEQ ID NO: 25;
b. a VH comprising an amino acid sequence having at least 80% sequence identity, such as at least 90%, at least 95%, at least 98%, or at least 99% sequence identity, to an amino acid sequence of SEQ ID NO: 26;
c. a VH comprising an amino acid sequence having at least 80% sequence identity, such as at least 90%, at least 95%, at least 98%, or at least 99% sequence identity, to an amino acid sequence of SEQ ID NO: 27;
d. a VH comprising an amino acid sequence having at least 80% sequence identity, such as at least 90%, at least 95%, at least 98%, or at least 99% sequence identity, to an amino acid sequence of SEQ ID NO: 28;
e. a VH comprising an amino acid sequence having at least 80% sequence identity, such as at least 90%, at least 95%, at least 98%, or at least 99% sequence identity, to an amino acid sequence of SEQ ID NO: 29;
f. a VH comprising an amino acid sequence having at least 80% sequence identity, such as at least 90%, at least 95%, at least 98%, or at least 99% sequence identity, to an amino acid sequence of SEQ ID NO: 30;
g. a VH comprising an amino acid sequence having at least 80% sequence identity, such as at least 90%, at least 95%, at least 98%, or at least 99% sequence identity, to an amino acid sequence of SEQ ID NO: 31;
h. a VH comprising an amino acid sequence having at least 80% sequence identity, such as at least 90%, at least 95%, at least 98%, or at least 99% sequence identity, to an amino acid sequence of SEQ ID NO: 32.

As understood by those skilled in the art, a correlation between two amino acid sequences or between two nucleotide sequences can be described by the parameter “sequence identity”. The percentage of the sequence identity between two sequences can be determined, for example, by using a mathematical algorithm. Non-limiting examples of such mathematical algorithms include the algorithm of Myers and Miller (1988) CABIOS 4:11-17, the local homology algorithm of Smith et al. (1981) Adv. Appl. Math. 2: 482, the homology comparison algorithm of Needleman and Wunsch (1970) J. Mol. Biol. 48: 443-453, the method for searching for homology of Pearson and Lipman (1988) Proc. Natl. Acad. Sci. 85: 2444-2448, and modified form of the algorithm of Karlin and Altschul (1990) Proc. Natl. Acad. Sci. USA 87: 2264, which is described in the algorithm of Karlin and Altschul (1993) Proc. Natl. Acad. Sci. USA 90: 5873-5877. By using programs based on such mathematical algorithms, sequence comparisons (that is, alignments) for determining sequence identity can be performed. The program may be suitably executed by a computer. Examples of such programs include, but are not limited to, CLUSTAL of the PC/Gene program, ALIGN program (Version 2.0), and GAP, BESTFIT, BLAST, FASTA, and TFASTA of the Wisconsin genetics software package. Alignments using the programs can be performed, for example, by using initial parameters.

In some embodiments of the anti-CTLA-4 single heavy-chain antibody of the present disclosure, the antibody may have an amino acid sequence set forth in any one of SEQ ID NOs: 33-40.

The present disclosure relates to a method for removing a regulatory T cell (Treg) and/or increasing the CD8+ T cell/Treg cell ratio in a subject, a method for treating refractory cancer, a method for treating cancer, and a method for reducing side effects of a CTLA4 antibody in treating cancer.

As used herein, the term “treatment” refers to a therapeutic treatment in which the objective is to reverse, alleviate, ameliorate, inhibit, slow; or stop the progression or severity of a condition associated with a disease or disorder. The term “treatment” includes reducing or alleviating at least one side effect or symptom of a disease or disorder. A treatment is generally “effective” if one or more symptoms or clinical markers are reduced. Alternatively, a treatment is “effective” if the progression of the disease is reduced or stopped, that is, “treatment” includes not only the amelioration of a symptom, but also the cessation, at least slowing, of the progression or worsening of a symptom that is expected in the absence of treatment. Beneficial or desired clinical outcomes include, but are not limited to, alleviation of one or more symptoms, diminishment of extent of disease, stabilized (that is, not worsening) state of disease, delay or slowing of disease progression, amelioration or remission of the disease state, and remission (whether partial or total), whether detectable or undetectable.

As used herein, the terms “subject”, “patient”, and “individual” are used interchangeably herein and refer to an animal, such as a human. The term subject also includes “non-human mammals”, such as, for example, rats, mice, rabbits, sheep, cats, dogs, cows, pigs, and non-human primates. In a preferred embodiment, the subject is a human subject.

In some embodiments of the method described above, the disease is cancer. Specific examples of cancers include, but are not limited to: basal cell carcinoma, bile duct cancer, bladder cancer, bone cancer, breast cancer, peritoneal cancer, cervical cancer, bile duct cancer, choriocarcinoma, colon and rectal cancer, connective tissue cancer, digestive system cancer, endometrial cancer, esophageal cancer, eye cancer, head and neck cancer, stomach cancer, glioblastoma, liver cancer, kidney cancer, laryngeal cancer, leukemia, lung cancer (e.g., small cell lung cancer, non-small cell lung cancer, lung adenocarcinoma, and lung squamous cell carcinoma), lymphoma, including Hodgkin lymphoma and non-Hodgkin lymphoma, melanoma, myeloma, neuroendocrine neoplasm (e.g., neuroblastoma), oral cancer, ovarian cancer, pancreatic cancer, prostate cancer, retinoblastoma, rhabdomyosarcoma, rectal cancer, respiratory system cancer, salivary gland cancer, sarcoma, skin cancer, squamous cell carcinoma, testicular cancer, thyroid cancer, uterine or endometrial cancer, urinary system cancer, B cell lymphoma, chronic lymphocytic leukemia (CLL), acute lymphoblastic leukemia (ALL), hairy cell leukemia, chronic myeloblastic leukemia, and the like. In a preferred embodiment, the cancer is selected from liver cancer, melanoma, non-small cell lung cancer, and advanced neuroendocrine neoplasm (NEN).

In some embodiments of the method described above, the refractory cancer is primary hepatocellular carcinoma (HCC) or castration-resistant prostate cancer (CRPC).

In some embodiments of the method described above, the method further comprises the step of administering one or more additional therapies. For example, in some embodiments, the therapy is selected from chemotherapy, radiation therapy, immunotherapy, and surgical therapy.

In some embodiments, the immunotherapy is selected from a therapy directed against an immune checkpoint molecule, a CAR-T cell therapy, and a CAR-NK cell therapy. For example, the immune checkpoint molecule may be selected from PD-1, PD-L1, PD-L2, CTLA4, OX40, LAG3, TIM3, TIGIT, and CD103.

In some embodiments, the chemotherapy is selected from a combination chemotherapy regimen comprising epirubicin, oxaliplatin, and fluorouracil.

### EXAMPLES

The present invention is further described with reference to the following specific examples, and the advantages and features of the present invention will be clearer as the description proceeds. These examples are illustrative only and do not limit the scope of the present invention in any way. It should be appreciated by those skilled in the art that modifications and replacements can be made to the details and form of the technical solutions of the present invention without departing from the spirit and scope of the present invention and that all these modifications and replacements fall within the scope of the present invention.

In the following experiments, the administration dose and mode, the Treg killing effect, the pharmacokinetic activity, and the anti-tumor effect of the anti-CTLA-4 single heavy-chain antibody were investigated taking the anti-CTLA-4 single heavy-chain antibody having an amino acid sequence set forth in SEQ ID NO: 33 as an example.

### Example 1. Dose Escalation and Dose Expansion of Anti-CTLA-4 Single Heavy-Chain Antibody

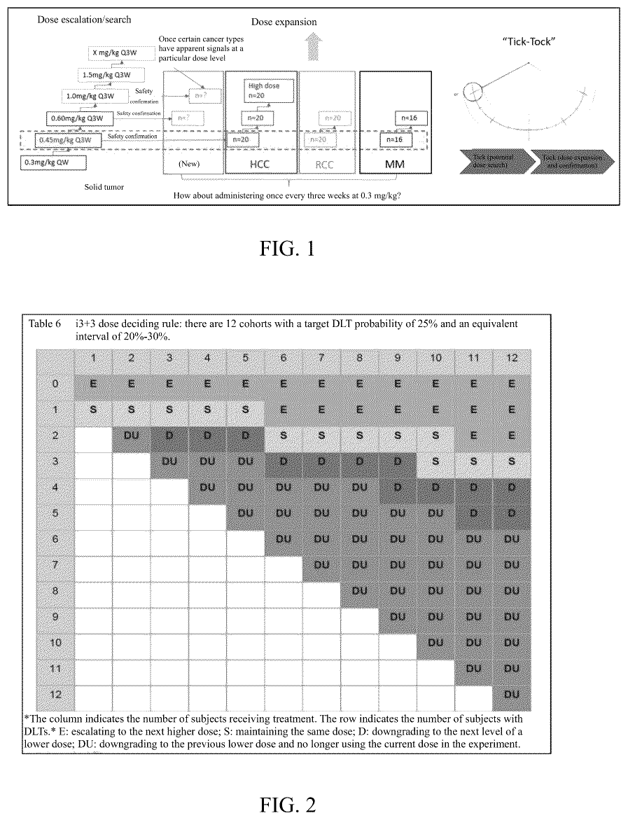

The example related to a phase I, open-label, and international multi-center study to evaluate the safety, tolerability, pharmacokinetics and anti-tumor activity of an anti-CTLA-4 single heavy-chain antibody in subjects with advanced solid tumors. The test is divided into 2 parts: “dose escalation” and “dose expansion”. Part I was a dose escalation stage. The decision of “upgrade”, “stay”, or “downgrade” was performed according to the i3+3 dose escalation algorithm (FIG. 1 and FIG. 2) and according to the combination of the dose limiting toxicity (DLT) and the comprehensive toxicity performance (including the late toxic reaction) at each dose level with the algorithm, while there was the possibility of reattempting unless the toxicity conditions exhibited by the cohort directly triggered DU (it would come to the previous dose level and the dose was not tried again).

The escalation method not only provides greater flexibility for escalation in clinical trials, but also is easy to operate by simple and visual charts, and compared with the traditional escalation method, the method optimizes the risk of “wrong estimation” by the aid of the underlying mathematical principle thereof, so that more accurate and fair safety assessment of each dose group cohort is guaranteed.

At the same time, in the test cohort, as the dose upgraded in the first part, an efficacy signal was expected to begin to occur in a certain dose group. Given the effector characteristics of immune drugs, multiple “clinically useful doses” often existed. Previous studies had suggested that the “optimal target dose” of the same drug for different types of tumors was not consistent (see, e.g., approved tumor species of ipilimumab and administration methods), and that the maximum tolerated dose (MTD) was not necessarily reached.

Thus, in addition to safety assessments, in the trial, according to the PD/PK characteristics and the efficacy profile (changes in tumor imageology, changes in tumor markers, and the like) exhibited in each dose group, evaluation was performed, and after regular scientific review and discussion mainly including clinicians to determine whether the dose “had a good benefit/risk ratio”, dose expansion in a specific tumor species could be initiated from a certain dose (Tock stage). At the same time, dose escalation was continued to further explore the safety margin of the drug and further determine the next dose had the potential to expand (with a good benefit/risk ratio) (Tick stage). If the “benefit/risk ratio” of a higher dose group was still deemed to be acceptable and had the potential to expand by the SRC mainly consisting of clinicians described above, the expansion in a specific tumor species would likewise be performed in the manner described above (Tock stage). Thus, like pendulum movement (Tick-Tock), the “benefit/risk ratio” evaluation was completed while the safety confirmation was continuously completed via the dose escalation in extensive solid tumors, and once confirmed, the dose was immediately pushed to the expansion part for POC verification in a selected “efficacy verification model” (a specific tumor species with potential); at the same time, the trial would continue into the next “pendulum cycle”.

Compared with the previous concept of “entering the next step after finding the MTD”, the method fully considered the factors of the characteristic of dose-effect diversification of immune drugs (“optimal dose levels” of different tumor species were different, and “onset doses” may also be different) and tumor species difference and the like, ensured the ethical safety of escalation, and accelerated the speed of entering dose expansion. Meanwhile, due to the setting of “pendulum mode”, each tumor species in the dose expansion stage would have data under different dose levels, so that the pharmacokinetic and efficacy characteristics and clinical benefit difference of “different tumor species under the same dose” and “the same tumor species under different doses” could be more comprehensively understood and analyzed. Furthermore, given the hypothesis that “sensitive onset doses for specific tumor species were different” that may exist, the flexibility of the expansion stage cohort setup was retained, allowing additional exploratory cohorts to be added according to the new signals found in any of the dose stages of the “pendulum mode” described above, thereby increasing the flexibility and compatibility provided by the test design for potential applications of the anti-CTLA-4 single heavy-chain antibody.

The dose escalation test of 2 cohorts, 19 patients in total, had been completed, wherein 7 persons were dosed once every week with 0.3 mg/kg, 6 persons were dosed once every three weeks with 0.6 mg/kg, and 6 persons were dosed once every three weeks with 0.45 mg/kg. The tumors involved included endometrial cancer, non-clear cell renal cell carcinoma, clear cell renal cell carcinoma, non-small cell lung cancer, head and neck cancer, breast cancer, castration-resistant prostate cancer, testicular cancer, urothelial cancer, liver cancer, esophageal cancer, mesothelioma, penile cancer, colorectal cancer, and the like.

In terms of safety, adverse events of the anti-CTLA-4 single heavy-chain antibody were concentrated in the digestive tract, mainly diarrhea (grade 2 or 3) or colitis (grade 1 or 2), compared with the adverse events reported of other CTLA-4 drugs involving multiple organs (the most common first 5 were rash, diarrhea or colitis, hepatitis, hypophysitis, pneumonia, and the like, respectively). The toxicity profile of the anti-CTLA-4 single heavy-chain antibody was narrower/more concentrated than that of the drug for the same target, and the existing data suggested that the gastrointestinal symptoms and pathological findings after the anti-CTLA-4 single heavy-chain antibody was exposed showed an obvious “separation” phenomenon compared with the reports of the drug for the same target: The G3 diarrhea was a simple watery diarrhea, and some patients were with G1-2 colitis without mucopurulent bloody stools and without abdominal pain, but the microscopic pathology was mostly mild, which was a phenomenon similar to the preclinical finding.

Enteroscopy and pathological biopsy prompted: In addition to 1 patient (occurring after 12 injections) who showed the typical ulcerative colitis, all other patients showed atypical manifestations (the report indicating “enteritis could not be excluded, possibly related”) or mild inflammatory cell activity under a biopsy scope, and the diarrhea symptoms of the patients described above could be rapidly relieved after drug intervention. The associated digestive tract comprehensive diagnosis (diarrhea/enteritis) could be well controlled and finally relieved by clinical use of anti-diarrhea drugs and a conventional dose of hormone (0.5 mg to 1 mg/kg prednisone equivalent).

In addition to the digestive tract-related adverse events, 3 cases of rash (≤ grade 2) were reported only in the administration dose group once every week with 0.3 mg/kg, whereas no skin-related adverse events were suggested in any of the other dose groups. No clear immune-related hepatitis, hypophysitis, pneumonia, and nephritis were observed. Grade 3 treatment-related adverse events (TRAEs) were all diarrhea, with no grade 4 or more TRAE occurring.

6 subjects were in the 0.45 mg/kg Q3W dose group, 0 with diarrhea, 0 with colitis, 0 with fatigue, 0 with rash, and 1 with pruritus. The overall tolerability was good.

Table 1 below shows a summary of adverse events in the 0.3 mg/kg QW and 0.6 mg/kg Q3W dose groups.

TABLE 1
Summary of adverse event results in each dose group

| | Anti-CTLA-4 single heavy-chain | | | Anti-CTLA-4 single heavy-chain | | |
|---|---|---|---|---|---|---|
| | antibody 0.3 mg/kg QW | | | antibody 0.6 mg/kg Q3W | | |
| | Grade | | | | | |
| | All grades | Grade 1/2 | ≥ Grade 3 | All grades | Grade 1/2 | ≥ Grade 3 |
| | N (%) | N (%) | N (%) | N (%) | N (%) | N (%) |
| Number of | 7 (100.0) | 7 (100.0) | 7 (100.0) | 6 (100.0) | 6 (100.0) | 6 (100.0) |
| subjects | | | | | | |
| Number of | 5 (71.4) | 5 (71.4) | 2 (28.6) | 4 (66.7) | 3 (50.0) | 1 (16.7) |
| subjects with | | | | | | |
| TRAE | | | | | | |
| Diarrhea | 3 (42.9) | 1 (14.3) | 2 (28.6) | 3 (50.0) | 2 (33.3) | 1 (16.7) |
| Colitis | 2 (28.6) | 2 (28.6) | 0 (0.0) | 1 (16.7) | 1 (16.7) | 0 (0.0) |
| Fatigue | 3 (42.9) | 3 (42.9) | 0 (0.0) | 0 (0.0) | 0 (0.0) | 0 (0.0) |
| Rash | 3 (42.9) | 3 (42.9) | 0 (0.0) | 0 (0.0) | 0 (0.0) | 0 (0.0) |
| Pruritus | 2 (28.6) | 2 (28.6) | 0 (0.0) | 0 (0.0) | 0 (0.0) | 0 (0.0) |

### Example 2. In Vitro and In Vivo Treg Killing Effect of Anti-CTLA-4 Single Heavy-Chain Antibody

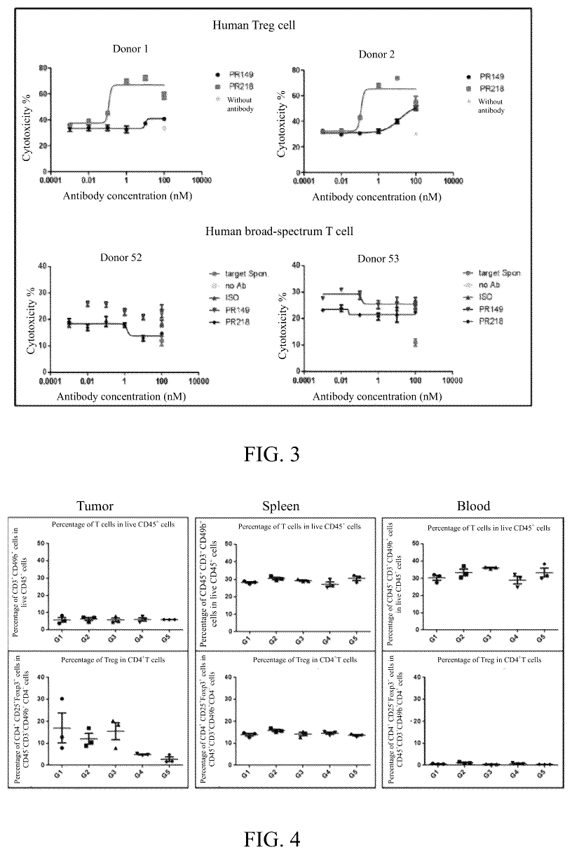

The human T_{reg} cell-clearing activity of the anti-CTLA-4 single heavy-chain antibody was evaluated in an in vitro ADCC killing assay. In the experiment, human T_{reg} cells were differentiated in vitro from undifferentiated CD4 T cells, then labeled with the calcein AM, followed by incubation with the antibody and primary human PBMCs for several hours. The numerical detection of calcein AM in the supernatant revealed that the anti-CTLA-4 single heavy-chain antibody (PR218) had potent T_{reg} cell killing activity compared to an ipilimumab mimic (PR149) (FIG. 3). In contrast, there was no apparent cell killing mediated by the anti-CTLA-4 single heavy-chain antibody in primary human total T cells.

To assess changes in tumor infiltrating cell populations in the efficacy model, another anti-CTLA-4 single heavy-chain antibody study was performed in the mouse model. Each of the C57BL/6 mice knocked in with the human CTLA-4 gene was inoculated subcutaneously with MC38 tumor cells (1×10⁶), and the mean tumor size at day 10 after the tumor inoculation was 373 mm³. Animals were randomized into groups and dosed with 10 mg/kg of human IgG1 (G1), 10 mg/kg of ipilimumab mimic (G2), 5.4 mg/kg of hIgG1 HCAb (PR271) (G3), and 5.4 mg/kg (G4) and 1.5 mg/kg of the anti-CTLA-4 single heavy-chain antibody (G5). The mice were given a second dose on day 3 and were euthanized 24 hours later, and tumor, spleen, and blood samples were collected for FACS analysis.

The primary endpoint was to determine the changes in the ratio of different immune cell populations in tumor, blood, and spleen in tumor-bearing mice after administration. The cell population differences between different groups were analyzed using a one-way repeated measures analysis of variance (ANOVA) method. The comparison was performed with the vehicle group using the Bonferroni's multiple comparison method. As shown in the results in FIG. 4 and Table 2, in the tumor sample, a decrease in T_{reg} proportion in CD4⁺ T cells was observed in the anti-CTLA-4 single heavy-chain antibody administration groups at a dose of 5.4 mg/kg or 1.5 mg/kg, while no change was observed when the ipilimumab mimic was used. No significant change in T_{reg} proportion in CD4⁺ cells was observed in spleen and blood samples when the anti-CTLA-4 single heavy-chain antibody or the ipilimumab mimic was used.

TABLE 2
Ratio of T cell to NK cell

| | Average ratio of T cell to NK cell | | | | | | |
|---|---|---|---|---|---|---|---|
| | | | CD4+ | CD8+ | | NK | NKT |
| Sample | Group | T cell | T cell | T cell | T_{reg} | cell | cell |
| Tumor | Group 1: hIgG (10 mg/kg) | 5.76 | 2.11 | 1.93 | 16.94 | 2.50 | 0.27 |
| | Group 2: ipilimumab mimic (10 mg/kg) | 6.29 | 2.22 | 2.33 | 12.05 | 2.14 | 0.32 |
| | Group 3: hIgG1 HCAb (5.4 mg/kg) | 5.79 | 1.85 | 2.47 | 15.50 | 1.67 | 0.29 |
| | Group 4: anti-CTLA-4 single heavy- | 6.01 | 2.20 | 2.05 | 4.88 | 2.01 | 0.28 |
| | chain antibody (5.4 mg/kg) | | | | | | |
| | Group 5: anti-CTLA-4 single heavy- | 5.95 | 1.80 | 2.40 | 2.74 | 2.80 | 0.33 |
| | chain antibody (1.5 mg/kg) | | | | | | |
| Spleen | Group 1: hIgG (10 mg/kg) | 28.28 | 15.32 | 9.17 | 13.93 | 2.24 | 0.80 |
| | Group 2: ipilimumab mimic (10 mg/kg) | 30.55 | 17.52 | 8.56 | 15.87 | 2.52 | 1.11 |
| | Group 3: hIgG1 HCAb (5.4 mg/kg) | 29.30 | 14.91 | 9.79 | 14.24 | 2.74 | 1.11 |
| | Group 4: anti-CTLA-4 single heavy- | 27.16 | 15.46 | 7.30 | 14.62 | 3.15 | 1.16 |
| | chain antibody (5.4 mg/kg) | | | | | | |
| | Group 5: anti-CTLA-4 single heavy- | 30.60 | 16.02 | 10.42 | 13.68 | 2.88 | 1.04 |
| | chain antibody (1.5 mg/kg) | | | | | | |
| Blood | Group 1: hIgG (10 mg/kg) | 30.35 | 18.45 | 10.73 | 0.60 | 3.49 | 0.19 |
| | Group 2: ipilimumab mimic (10 mg/kg) | 33.39 | 19.77 | 11.41 | 1.15 | 4.40 | 0.47 |
| | Group 3: hIgG1 HCAb (5.4 mg/kg) | 36.06 | 20.39 | 13.21 | 0.49 | 3.85 | 0.58 |
| | Group 4: anti-CTLA-4 single heavy- | 29.03 | 17.43 | 9.14 | 0.72 | 4.81 | 0.60 |
| | chain antibody (5.4 mg/kg) | | | | | | |
| | Group 5: anti-CTLA-4 single heavy- | 33.35 | 18.79 | 12.05 | 0.39 | 4.18 | 0.56 |
| | chain antibody (1.5 mg/kg) | | | | | | |

### Example 3. Pharmacokinetic Results of Anti-CTLA-4 Single Heavy-Chain Antibody

Pharmacokinetics of Anti-CTLA-4 Single Heavy-Chain Antibody in Female C57BL/6 Mice after a Single Intravenous Administration

The objective of the study was to determine the PK characteristics of the anti-CTLA-4 single heavy-chain antibody (PR218) in female C57BL/6 mice after an intravenous administration. 6 SPF-grade C57BL/6 mice (female) were dosed with 1 and 5 mg/kg of the anti-CTLA-4 single heavy-chain antibody (N=3), respectively. Blood and serum samples were collected at the following time points: before administration, 0.083 hrs, and days 1, 2, 4, 7, 10, and 14. The concentration of the anti-CTLA-4 single heavy-chain antibody in the serum of the C57BL/6 mice was determined by ELISA. PR218 PK parameters (t_{1/2}, Cₘₐₓ, total clearance [CL], volume of distribution at steady state [Vss], mean residence time [MRT], and area under the concentration-time curve [AUC]) in serum were determined from the mean concentration-time data. Parameters were estimated using a non-compartmental analysis method (WinNonlin® Professional 6.4, Pharsight, Mountain View, USA). Any values below a quantifiable level were excluded in PK parameter calculations using WinNonlin.

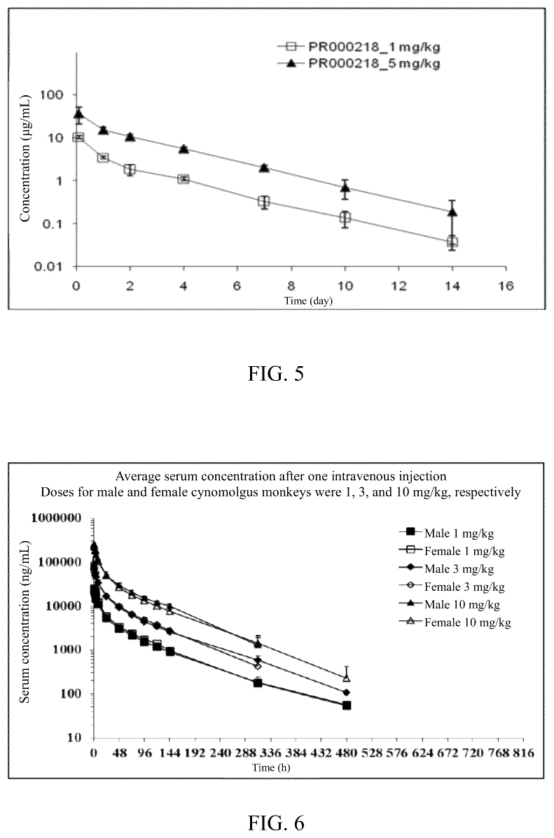

FIG. 5 is a diagram showing mean serum concentration-time curves of the anti-CTLA-4 single heavy-chain antibody after intravenous administration at doses of 1 mg/kg and 5 mg/kg. The serum concentration of the anti-CTLA-4 single heavy-chain antibody was increased with the increase of the dose and were biphasic over time.

In female C57BL/6 mice, after an intravenous administration of 1 mg/kg of the anti-CTLA-4 single heavy-chain antibody, t_{1/2}, Cₘₐₓ, the area under the concentration-time curve between time 0 to the last measurable concentration (AUCₗₐₛₜ), AUC_{INF}, CL_{Z}, MRTₗₐₛₜ, and Vₛₛ were 2.22±0.0281 days, 10.4±0.758 μg/mL, 16.1±1.48 day*μg/mL, 16.2±1.52 day*μg/mL, 62.1±58.0 mL/kg/day, 2.05±0.228 days, and 134±12.1 mL/kg, respectively (Table 3).

In female C57BL/6 mice, after an intravenous administration of 5 mg/kg of the anti-CTLA-4 single heavy-chain antibody, t_{1/2}, Cₘₐₓ, AUCₗₐₛₜ, AUC_{INF}, CL_{Z}, MRTₗₐₛₜ, and Vₛₛ were 1.94±0.718 days, 36.6±15.9 μg/mL, 73.3±15.3 day*μg/mL, 74.0±15.9 day*μg/mL, 69.8±15.4 mL/kg/day, 2.41±0.144 days, and 176±40.5 mL/kg, respectively (Table 3).

The terminal elimination half-life and CLz remained constant at doses ranging from 1 mg/kg to 5 mg/kg, with average values of approximately 2.1 days and 66.0 mL/kg/day, respectively. Total exposure measured using AUC_{INF} as a parameter showed an increase in proportion to the dose in the range of 1 mg/kg to 5 mg/kg. The data indicate that the PK of the anti-CTLA-4 single heavy-chain antibody in mice was linear over an intravenous dose range of 1 mg/kg to 5 mg/kg.

TABLE 3
Pharmacokinetic parameters of anti-CTLA-4 single
heavy-chain antibody in female C57BL/6 mice

| PK | | 1 mg/kg | | 5 mg/kg | |
|---|---|---|---|---|---|
| parameter | Unit | Mean | SD | Mean | SD |
| t_{1/2} | Day | 2.22 | 0.0281 | 1.94 | 0.718 |
| Cₘₐₓ | μg/mL | 10.4 | 0.758 | 36.6 | 15.9 |
| AUCₗₐₛₜ | Day*μg/mL | 16.1 | 1.48 | 73.3 | 15.3 |
| AUC_{INF} | Day*μg/mL | 16.2 | 1.52 | 74.0 | 15.9 |
| CL_{Z} | mL/kg/day | 62.1 | 5.80 | 69.8 | 15.4 |
| MRTₗₐₛₜ | Day | 2.05 | 0.228 | 2.41 | 0.144 |
| V_{SS} | mL/kg | 134 | 12.1 | 176 | 40.5 |

Pharmacokinetic Study of Anti-CTLA-4 Single Heavy-Chain Antibody after Single Intravenous Bolus Administration in Cynomolgus Monkeys

The PK of the anti-CTLA-4 single heavy-chain antibody was evaluated in cynomolgus monkeys after single intravenous bolus administration of the anti-CTLA-4 single heavy-chain antibody at doses of 1, 3, and 10 mg/kg. A total of 18 cynomolgus monkeys (9 males and 9 females) were randomly divided into 3 groups and tested at each dose level.

Blood samples for PK assessment were collected at the following time points: before administration and 0.033, 0.5, 2, 4, 8, 24, 48, 72, 96, 120, 144, 312, 480, 648, and 816 hrs (day 35) after administration. The serum concentration of the anti-CTLA-4 single heavy-chain antibody was determined by a validated ELISA method. The PK parameters (t_{1/2}, Cₘₐₓ when C₀, CL, Vₛₛ, and AUC) of the anti-CTLA-4 single heavy-chain antibody in serum were determined from mean concentration-time data. The parameters were estimated using non-compartmental analysis.

To detect anti-drug antibodies (ADA) generated after administration of the anti-CTLA-4 single heavy-chain antibody, blood samples were collected at the following time points for serum extraction: before administration and 312 hrs (day 14), 480 hrs (day 21), and 816 hrs (day 35) after administration. The ADA concentration was detected using a validated electrochemiluminescence method.

Before administration, all animals did not show any detectable ADA. On day 14 after administration, most animals showed ADA negative, but 1 of the 3 females in the 1 mg/kg group, 2 of the 3 females in the 3 mg/kg group, and all 3 males and 1 of the 3 females in the 10 mg/kg group were exceptions, which showed ADA positive, but the titers were relatively low. On day 21, 1 of the 3 females in the 1 mg/kg group, 2 of the 3 males and 2 of 3 females in the 3 mg/kg group, and all 3 males and 1 of the 3 females in the 10 mg/kg group showed ADA positive, but the titers were relatively low. On day 35, the ADA positive rate increased, with 1 of the 3 males and 2 of the 3 females in the 1 mg/kg group, 2 of the 3 males and all females in the 3 mg/kg group, and all 3 males and 2 of the 3 females in the 10 mg/kg group, respectively, showing ADA positive.

FIG. 6 is a diagram showing mean serum concentration-time curves of the anti-CTLA-4 single heavy-chain antibody after intravenous administration at single doses of 1, 3, and 10 mg/kg in cynomolgus monkeys.

After the cynomolgus monkeys were intravenously dosed with the anti-CTLA-4 single heavy-chain antibody at doses of 1, 3, and 10 mg/kg, Cmax reached at initial time point C₀ (concentration measured immediately after first injection) were 25.2±2.33 μg/mL, 82.9±12.6 μg/mL, and 244±22.9 g/mL, respectively; t1/2 was 3.02±0.490 days, 2.77±0.482 days, and 2.55±0.412 days, respectively; AUCₗₐₛₜ was 26.2±3.74 μg*day/mL, 75.9±4.48 μg*day/mL, and 231±23.0 μg*day/mL, respectively; AUC_{INF WAS} 26.6±3.71 μg*day/mL, 76.8±4.69 μg*day/mL, and 233±23.2 μg*day/mL, respectively; CL was 38.4±6.05 mL/day/kg, 39.2±2.52 mL/day/kg, and 43.3±4.38 mL/day/kg, respectively; Vd was 112±14.3 mL/kg, 109±8.67 mL/kg, and 118±19.3 mL/kg, respectively (Table 4).

Among the 1, 3, and 10 mg/kg dose groups, t_{1/2} and CL remained constant. In the dose range of 1 mg/kg to 10 mg/kg, C₀ and the total exposure measured using AUC_{INF} as a parameter showed an increase in proportion to the dose. The data indicate that the PK of the anti-CTLA-4 single heavy-chain antibody in cynomolgus monkeys was linear over an intravenous dose range of 1 mg/kg to 10 mg/kg.

TABLE 4
Pharmacokinetic parameters of anti-CTLA-4 single heavy-
chain antibody in male and female cynomolgus monkeys

| | Intravenous administration of anti-CTLA-4 single heavy-chain | | | | | |
|---|---|---|---|---|---|---|
| | antibody | | | | | |
| | 1 mg/kg (N = 6) | | 3 mg/kg (N = 6) | | 10 mg/kg (N = 6) | |
| PK parameter | Mean | SD | Mean | SD | Mean | SD |
| C₀ (μg/mL) | 25.2 | 2.33 | 82.9 | 12.6 | 244 | 22.9 |
| t_{1/2} (day) | 3.02 | 0.490 | 2.77 | 0.482 | 2.55 | 0.412 |
| Vd (mL/kg) | 112 | 14.3 | 109 | 8.67 | 118 | 19.3 |
| CL | 38.4 | 6.05 | 39.2 | 2.52 | 43.3 | 4.38 |
| (mL/day/kg) | | | | | | |
| AUCₗₐₛₜ | 26.2 | 3.74 | 75.9 | 4.48 | 231 | 23.0 |
| (μg*day/mL) | | | | | | |
| AUC_{inf} | 26.6 | 3.71 | 76.8 | 4.69 | 233 | 23.2 |
| (μg*day/mL) | | | | | | |

### Example 4. Pharmacokinetic Results of Anti-CTLA-4 Single Heavy-Chain Antibody in Patients

### Drug Information

The anti-CTLA-4 single heavy-chain antibody injection contained 4.0 mL of a drug liquid with 80 mg of the anti-CTLA-4 single heavy-chain antibody (having an amino acid sequence set forth in SEQ ID NO: 33) in each bottle, and the injection was administered on the same day. The dose was calculated by body weight. The injection was stored at 2-8° C., and the drug was stored vertically by using the original package. The formulating mode was as follows: extracting the anti-CTLA-4 single heavy-chain antibody with a required dose from a bottle, and adding the anti-CTLA-4 single heavy-chain antibody into a infusion bag with a 0.9% sodium chloride injection or 5% glucose injection for dilution, wherein the dilution concentration range was 0.12 mg/mL to 10.0 mg/mL.

The diluent was required to be infused intravenously for more than 90 minutes, but the time should not exceed 4 hours, namely the infusion time was controlled between 90 minutes and 4 hours. If the anti-CTLA-4 single heavy-chain antibody injection was formulated into 50 mL of a diluent, the dripping speed should be controlled to be 0.21 mL/min to 0.56 mL/min, and if the anti-CTLA-4 single heavy-chain antibody injection was formulated into 100 mL of a diluent, the dripping speed should be controlled to be 0.42 mL/min to 1.1 mL/min. During intravenous drip, a sterile intravenous drip apparatus comprising an online filter membrane of 0.2 microns without pyrogen and having low protein tuberculosis was adopted, and after intravenous infusion, 20 mL of a 0.9% sodium chloride injection or 5% glucose injection was used for flushing the pipeline of the intravenous drip apparatus.

### Administration Information

A portion of cancer patients were divided into three cohorts and infused with the anti-CTLA-4 single heavy-chain antibody injection: the first cohort of patients was designed to be dosed with 0.3 mg/kg once every week: the second cohort of patients was designed to be dosed with 0.6 mg/kg once every three weeks; the third cohort of patients was designed to be dosed with 0.45 mg/kg once every three weeks.

Another portion of cancer patients were divided into 2 cohorts and infused with ipilimumab: the first cohort of patients was designed to be dosed with 0.3 mg/kg once every three weeks; the second cohort of patients was designed to be dosed with 3 mg/kg once every three weeks.

### Data Acquisition

Peripheral blood was collected before and at various time points after one cycle of administration to patients (Australia patients) to determine the pharmacokinetic (PK) data and the concentration of the anti-CTLA-4 single heavy-chain antibody in serum as well as the anti-drug antibody (ADA).

The concentration of the anti-CTLA-4 single heavy-chain antibody in human serum was determined by adopting a fully validated electrochemiluminescence method (ECL). The lower limit of quantification (LLOQ) of the analysis method was 20.5 ng/mL, the upper limit of quantification was 5000.0 ng/mL, and samples exceeding the upper limit of quantification could be diluted up to 400-fold. The sample had good stability under room temperature, refrigeration, and freezing conditions.

A fully validated bridged electrochemiluminescence method (ECL) was used to analyze the anti-drug antibody (ADA), and a multi-level analysis method was used, namely firstly carrying out screening tests on all samples, secondly carrying out confirmation tests on the specificity of the suspected antibody positive samples, and carrying out titer tests on the samples with determined antibody positive. The sensitivity of the analysis method was 34.0 ng/mL. The drug was well tolerable at the drug concentration level of the samples collected.

The PK parameters were estimated using a non-compartmental analysis method.

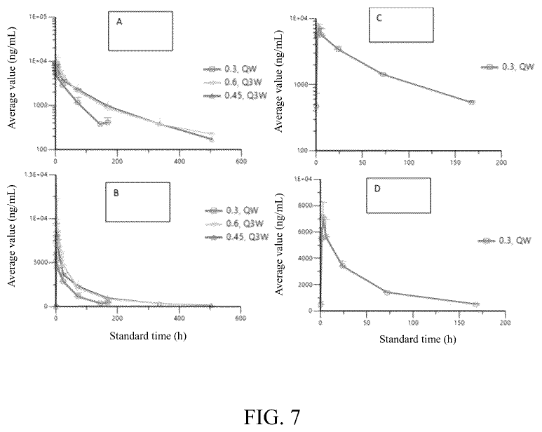

The concentration of the anti-CTLA-4 single heavy-chain antibody in serum-time curves for various dose levels of the anti-CTLA-4 single heavy-chain antibody tested in subjects with advanced solid tumors are shown in FIG. 7, and a PK parameter summary is shown in Table 5.

TABLE 5
Pharmacokinetic parameters of anti-CTLA-4 single heavy-chain antibody in subjects

| | 0.3 mg/kg QW | | 0.6 mg/kg Q3W | 0.45 mg/kg Q3W | ipilimumab | ipilimumab |
|---|---|---|---|---|---|---|
| Parameter | C1D1 (n = 7) | C1D22 (n = 3) | Cycle (n = 6) | Cycle 1 (n = 3) | (0.3 mg/kg Q3W) | (3 mg/kg Q3W) |
| Cₘₐₓ (ug/mL) | 6.01₍₁₈₎ | 7.25₍₁₄₎ | 8.48₍₄₆₎ | 8.63₍₁₀₎ | 7.3 | 76.1 |
| (CV %) | | | | | | |
| Cₘᵢₙ (ug/mL) | 0.399₍₂₉₎ | 0.537_{(0.514~0.562)} | 0.228₍₁₁₎ | 0.172 | 1.04 | 9.97 |
| (CV %) | | | | | | |
| AUC₀₋ₜₐᵤ | 24 ₍₂₂₎ | 299_{(284~315)} | 687₍₁₉₎ | 511_{(472~552)} | 1600 | 16100 |
| (ug*h/mL) (CV%) | | | | | | |

| | | | | | |
|---|---|---|---|---|---|
| t_{1/2} (day) (SD) | 2.05₍₁₈₎ | 2.22_{(2.06~2.40)} | 4.43₍₃₃₎ | 3.30_{(2.51~4.34)} | 14.7 |

PK properties observed in subjects with advanced solid tumors were similar to those predicted preclinically:

The half-life was short (about 2 to 4 days), so that the accumulation was small after multiple administrations: in comparison, the half-life of ipilimumab was 14.7 days:
the exposure (AUC) was close to the predicted value and was far lower than the exposure of ipilimumab under the same dose;
AUC increased with the increase of dose, substantially proportional to dose;
in addition, the data showed that the anti-CTLA-4 single heavy-chain antibody was low in immunogenicity: only one of the 13 patients was detected ADA positivity at one visit with a titer of 2.

### Example 5. Anti-Tumor Activity of Anti-CTLA-4 Single Heavy-Chain Antibody

### Drug Information

The anti-CTLA-4 single heavy-chain antibody injection contained 4.0 mL of a drug liquid with 80 mg of the anti-CTLA-4 single heavy-chain antibody (having an amino acid sequence set forth in SEQ ID NO: 33) in each bottle, and the injection was administered on the same day. The dose was calculated by body weight. The injection was stored at 2-8° C., and the drug was stored vertically by using the original package. The formulating mode was as follows: extracting the anti-CTLA-4 single heavy-chain antibody with a required dose from a bottle, and adding the anti-CTLA-4 single heavy-chain antibody into a infusion bag with a 0.9% sodium chloride injection or 5% glucose injection for dilution, wherein the dilution concentration range was 0.12 mg/mL to 10.0 mg/mL.

The diluent was required to be infused intravenously for more than 90 minutes, but the time should not exceed 4 hours, namely the infusion time was controlled between 90 minutes and 4 hours. If the anti-CTLA-4 single heavy-chain antibody injection was formulated into 50 mL of a diluent, the dripping speed should be controlled to be 0.21 mL/min to 0.56 mL/min, and if the anti-CTLA-4 single heavy-chain antibody injection was formulated into 100 mL of a diluent, the dripping speed should be controlled to be 0.42 mL/min to 1.1 mL/min. During intravenous drip, a sterile intravenous drip apparatus comprising an online filter membrane of 0.2 microns without pyrogen and having low protein tuberculosis was adopted, and after intravenous infusion, 20 mL of a 0.9% sodium chloride injection or 5% glucose injection was used for flushing the pipeline of the intravenous drip apparatus.

### Anti-Tumor Evaluation

Tumor patients were infused with the anti-CTLA-4 single heavy-chain antibody injection, 2 times of anti-tumor evaluations were carried out in the tumor patients, and in each anti-tumor evaluation, the tumor patients were divided into three cohorts for administration: the first cohort of patients was designed to be dosed with 0.3 mg/kg once every week: the second cohort of patients was 0.6 mg/kg once every three weeks; the third cohort of patients was 0.45 mg/kg once every three weeks.

In terms of clinical efficacy, 11 patients completed the first tumor evaluation, of which 9 maintained stable disease (SD). A 22% reduction in tumor from a baseline was observed in HCC patients, and AFP decreased to a normal level. CRPC patients achieved “PSA alleviation”. PSA decreased from a baseline of 210 μg/L by more than 50% to 91 μg/L and had remained so far (nearly 8 months). A recent review showed that PSA remained at 78 μg/L, and the patients felt good. A 6-14% tumor regression was observed in two EC and PRCC patients. 7 patients completed the second tumor evaluation, of which 2 maintained stable disease (SD), 1 had an alleviated disease (PR, tumor decreased by about 50% from a baseline, AFP had maintained normal so far), and 1 RCC patient with significant regression of part of the target lesion observed. The therapeutic results for the anti-CTLA-4 single heavy-chain antibody are summarized in Table 6.

TABLE 6
Anti-Tumor Effect of Anti-CTLA-4 Single Heavy-Chain Antibody

| | | Number of | | | | |
|---|---|---|---|---|---|---|
| | | patients | Number | Number | Number | |
| | Dose level | evaluated | of PR | of SD | of PD | Test result |
| First tumor | 0.3 MG/KG | 6 | 0 | 5 | 1 | Endometrial cancer (EC) was |
| evaluation | QW | | | | | reduced by 14% |
| | | | | | | PRCC was reduced by 14% |
| | 0.6 MG/KG | 4 | 0 | 3 | 1 | Prostate cancer (CRPC): PSA |
| | Q3W | | | | | was reduced from a baseline of |
| | | | | | | 210 MG/L to 91 MG/L |
| | 0.45 MG/KG | 1 | 0 | 1 | — | AFP of hepatocellular |
| | Q3W | | | | | carcinoma (HCC) patients was |
| | | | | | | reduced from 170 μG/L to 5 |
| | | | | | | μG/L, and the tumor was |
| | | | | | | reduced by 22% from a |
| | | | | | | baseline to normal level |
| Second | 0.3 MG/KG | 4 | 0 | 1 | 3 | |
| tumor | QW | | | | | |
| evaluation | 0.6 MG/KG | 2 | 0 | 1 | 1 | Kidney cancer (RCC): SD |
| | Q3W | | | | | (TL3 was reduced by 38%, |
| | | | | | | and TL5 was increased by |
| | | | | | | 48%) |
| | 0.45 MG/KG | 1 | 1 | — | — | AFP of hepatocellular |
| | Q3W | | | | | carcinoma (HCC) patients was |
| | | | | | | reduced to normal range (7 |
| | | | | | | μG/L), and the tumor was |
| | | | | | | reduced by 48.9% from a |
| | | | | | | baseline to normal level |

### Example 6. Therapeutic Results of Anti-CTLA-4 Single Heavy-Chain Antibody in Typical Patients

### Patient 1

Cancer patient 1 was intravenously infused with the anti-CTLA-4 single heavy-chain antibody, and the information of the patient is shown in Table 7 below. The patient had previously received surgery and radiation therapy and was previously dosed with lenvatinib, sorafenib, and SHR1701-001 (2020 Sep. 8, Hengrui PD-1; PD), and the reason for drug withdrawal was unknown. In addition, the patient had previously received PD-1/PD-L1 immunotherapy.

TABLE 7
Information of patient 1

| | Patient 1 |
|---|---|
| Sex | Male (Asian) |
| Age | 64 |
| Diagnosis | HCC diagnosed on Jul. 20, 2010 |
| Tumor type | TNM = T? N? M1 clinical phase IV |
| Previous disease | Chronic hepatitis B; frontal lobe cavernous |
| | hemangioma; mild duodenitis; gastroesophageal |
| | reflux disease; mild hearing loss in the right ear; |
| | moderate anemia; moderate thrombocytopenia; |
| | skin dryness |
| Baseline | Mild lymphopenia, thrombocytopenia, moderate |
| hematology | anemia |
| Baseline | Moderate AST and ALT elevations without |
| biochemistry | jaundice; hypoalbuminemia |
| Previous cancer | None |
| Baseline tumor | Sum of total target lesions (right upper liver, |
| evaluation | right kidney) |
| | Non-target: left pleural effusion; multiple |
| | liver metastases; multiple lymph node metastases |
| Tumor | The right upper liver lesion reduced from 15 cm |
| evaluation at | to 8 cm by about 47% |
| week 6 | The right kidney reduced from 6 cm to 3.6 cm by |
| | about 40% |
| | Two intrahepatic metastases/original lesions |
| | increased significantly from 2-2.5 cm and |
| | 3.4-4.4 cm |

Patient 1 was infused with the anti-CTLA-4 single heavy-chain antibody injection containing 4.0 mL of a drug liquid with 80 mg of the anti-CTLA-4 single heavy-chain antibody (sequence set forth in SEQ ID NO: 33) in each bottle, and the injection was administered on the same day. The dose was calculated by body weight. The injection was stored at 2-8° C., and the drug was stored vertically by using the original package. The formulating mode was as follows: extracting the anti-CTLA-4 single heavy-chain antibody with a required dose from a bottle, and adding the anti-CTLA-4 single heavy-chain antibody into a infusion bag with a 0.9% sodium chloride injection or 5% glucose injection for dilution, wherein the dilution concentration range was 0.12 mg/mL to 10.0 mg/mL.

The diluent was required to be infused intravenously for more than 90 minutes, but the time should not exceed 4 hours, namely the infusion time was controlled between 90 minutes and 4 hours. If the anti-CTLA-4 single heavy-chain antibody injection was formulated into 50 mL of a diluent, the dripping speed should be controlled to be 0.21 mL/min to 0.56 mL/min, and if the anti-CTLA-4 single heavy-chain antibody injection was formulated into 100 mL of a diluent, the dripping speed should be controlled to be 0.42 mL/min to 1.1 mL/min. During intravenous drip, a sterile intravenous drip apparatus comprising an online filter membrane of 0.2 microns without pyrogen and having low protein tuberculosis was adopted, and after intravenous infusion, 20 mL of a 0.9% sodium chloride injection or 5% glucose injection was used for flushing the pipeline of the intravenous drip apparatus.

Patient 1 was dosed at 0.45 mg/kg once every three weeks for three times.

The indicator traits of the patients after infusion of the anti-CTLA-4 single heavy-chain antibody are shown in Tables 8-9.

TABLE 8
Lesion indicator of patient 1

| | Position | Baseline | 6 weeks | 12 weeks | 16 weeks |
|---|---|---|---|---|---|
| Target | Right | 14.5*10 cm | 12*8 cm | 8.5*4.5 cm | 7.5*4.0 cm |
| lesion | upper | | | | |
| | liver | | | | |
| | Right | 8*7 cm | 3*5.5 cm | 3*2 cm | 3*2 cm |
| | kidney | | | | |
| Change from | | NA | 22% | 48.9% | 53.3% |
| baseline | | | | | |
| Non- | Lung | Present | Not | Not present | Present |
| target | | | present | | |
| | Liver | Present | Present | Not present | Not present |
| | LN | Present | Not | Not present | Present |
| | | | determined | | |

| | | | |
|---|---|---|---|
| Overall | SD | PR | PR |
| response | | | |

TABLE 9
Other indicators of patient 1

| Indicator | 2020-10 | 2020-11 | 2020-12 | 2021-1 | 2021-2 |
|---|---|---|---|---|---|
| AFP | 170 | 5 | 5 | 7 | 9 |

| | |
|---|---|
| Biochemistry | Liver function was elevated; G3 total bilirubin |
| | was elevated |
| Liver biopsy | “The characteristics were consistent with chronic |
| | hepatitis . . . inflammation traits was not completely |
| | specific, and differential diagnosis included drug- |
| | induced liver injury (including autoimmune |
| | characteristics) and autoimmune hepatitis and |
| | infection . . . recommended auxiliary examination . . . ” |

As can be seen from Tables 8 and 9, after infusion of the anti-CTLA-4 single heavy-chain antibody, the tumor of patient 1 was significantly reduced, the change from baseline of the tumor was over 50%, the tumor biomarkers were significantly reduced, and liver function was significantly restored.

### Patient 2

Cancer patient 2 was intravenously infused with the anti-CTLA-4 single heavy-chain antibody, and the information of the patient is shown in Table 10 below. The patient had previously received prostatectomy (Feb. 15, 2001) and no radiation treatment, had 10 previously undergone Docetaxel, Cabazitaxel, Cosudex, and Zoladex chemotherapy, had not received PD-1/PD-L1 immunotherapy, and had been taking digoxin, rivaroxaban, verapamil, a calcium salt, vitamin D, denosumab, and dithiazide drugs.

TABLE 10
Information of patient 2

| | Patient 2 |
|---|---|
| Sex | Male |
| Age | 80 |
| Diagnosis | Prostate invasive adenocarcinoma (well-differentiated) |
| Tumor type | TNM = Tx Nx M1 clinical phase IV |
| Previous cancer | None |
| Previous disease | Osteoporosis; prostate cancer; anticoagulant therapy; |
| | atrial fibrillation; hypertension; bone metastasis; |
| | fluid retention; orthostatic syncope; sprain of right |
| | ankle; fracture of right arm |
| Baseline tumor | Total target lesion (right axilla) was 30 mm; bone |
| evaluation | metastasis |
| First tumor | PR |
| evaluation | |
| Second tumor | SD |
| evaluation | |

Patient 2 was infused with the anti-CTLA-4 single heavy-chain antibody injection containing 4.0 mL of a drug liquid with 80 mg of the anti-CTLA-4 single heavy-chain antibody in each bottle, and the injection was administered on the same day. The dose was calculated by body weight. The injection was stored at 2-8° C., and the drug was stored vertically by using the original package.

The formulating mode was as follows: extracting the anti-CTLA-4 single heavy-chain antibody with a required dose from a bottle, and adding the anti-CTLA-4 single heavy-chain antibody into a infusion bag with a 0.9% sodium chloride injection or 5% glucose injection for dilution, wherein the dilution concentration range was 0.12 mg/mL to 10.0 mg/mL.

The diluent was required to be infused intravenously for more than 90 minutes, but the time should not exceed 4 hours, namely the infusion time was controlled between 90 minutes and 4 hours. If the anti-CTLA-4 single heavy-chain antibody injection was formulated into 50 mL of a diluent, the dripping speed should be controlled to be 0.21 mL/min to 0.56 mL/min, and if the anti-CTLA-4 single heavy-chain antibody injection was formulated into 100 mL of a diluent, the dripping speed should be controlled to be 0.42 mL/min to 1.1 mL/min. During intravenous drip, a sterile intravenous drip apparatus comprising an online filter membrane of 0.2 microns without pyrogen and having low protein tuberculosis was adopted, and after intravenous infusion, 20 mL of a 0.9% sodium chloride injection or 5% glucose injection was used for flushing the pipeline of the intravenous drip apparatus.

Patient 2 was dosed at 0.6 mg/kg once every three weeks for three times.

The indicator traits of the patients after administration of the anti-CTLA-4 single heavy-chain antibody are shown in Tables 11-12.

TABLE 11
Lesion indicator of patient 2

| | Position | Baseline | 6 weeks | 12 weeks | 9 months |
|---|---|---|---|---|---|
| Target lesion | Lymph | 30 mm | 30 mm | 30 mm | 23 mm |
| | node | | | | |
| | Adrenal | 15 mm | 15 mm | 15 mm | 18 mm |
| | gland | | | | |
| Non-target | Bone | Present | Present | Present | Present |

| | |
|---|---|
| Overall response | SD had PSA response (decreased over 50%) |

TABLE 12
Other indicators of patient 2

| Clinical symptom | Aggravation of G2 diarrhea | | | |
|---|---|---|---|---|
| Endoscopy | Diagnosed immune-related colitis | | | |
| Biopsy | A. ileal biopsy-no obvious abnormalities. | | | |
| | B. caecum biopsy-mild focal colitis. | | | |
| | C. flexibility biopsy of liver-focal active colitis. | | | |
| | D. sigmoid biopsy-focal active colitis. | | | |
| | E. rectal biopsy-active colitis with increased apoptosis | | | |
| Biomarker | 2020-5 | 2020-6 | 2020-12 | 2021-2 |
| PSA | 240 | 92 | 89 | 78 |

As can be seen from Tables 11 and 12, after infusion of the anti-CTLA-4 single heavy-chain antibody, the tumor of patient 2 was significantly reduced, the change from baseline of the tumor was over 50%, and the tumor biomarkers were significantly reduced.

In summary, in combination with clinical data and observations, the overall clinical manifestations of the anti-CTLA-4 single heavy-chain antibody included: the tolerability of a patient to the anti-CTLA-4 single heavy-chain antibody was good; the generated adverse events conformed to the expected toxicity of the same target; compared with drugs with a similar target, the toxicity profile was more concentrated in the digestive tract system, and the adverse events occurred less frequently in organs such as skin, liver, kidney, lung, and the like; no serious specific pathological changes of the digestive system and relevant clinical risks (the risk of perforation of the digestive tract, serious injury of intestinal mucosa, and the like) were observed. Distinct anti-tumor effects were observed in the different dose groups including the initial dose, and a “significant clinical benefit” (“alleviated disease”: “alleviated tumor marker PSA”, and the like) consistent with current guidelines was observed in some refractory tumor species (e.g., cold tumor “CRPC” after failure of multi-line therapy: end-line HCC after failure of multi-line therapy) and had continued until now.

The half-life of the anti-CTLA-4 single heavy-chain antibody was short (about 2-4 days, relative to 14.7 days of ipilimumab), and thus the accumulation after multiple administrations was small. The exposure (AUC) was close to the predicted value and was far lower than the exposure of ipilimumab under the same dose. AUC increased with the increase of dose. Surprisingly, with relatively short half-life and relatively low drug accumulation and exposure, long-lasting changes in PD markers (increase of CD8 T cell/Treg ratio, ICOS+ T cells, and Ki67+ T cells) and significant anti-tumor effects were still observed in patients. This finding supports Q3W administration of the anti-CTLA-4 single heavy-chain antibody, and also indicates that the anti-CTLA-4 single heavy-chain antibody can maintain reliable long-term efficacy while having a short half-life to reduce drug exposure, and has the potential for clinical application to further develop long-cycle administration or PRN administration.

### REFERENCES

1. Aya F, Gaba L, Victoria I, et al. 2016. “Ipilimumab after progression on anti-PD-1 treatment in advanced melanoma.” Future Oncology 12(23): 2683-2688.
2. Banerjee, Kasturi et al. 2018. “Emerging Trends in the Immunotherapy of Pancreatic Cancer.” Cancer Letters 417: 35-46.
3. Bowyer S. et al. 2016. “Efficacy and toxicity of treatment with the anti-CTLA-4 antibody ipilimumab in patients with metastatic melanoma after prior anti-PD-1 therapy.” Br. J. Cancer 114(10):1084-1089.
4. Dogan, V., T. Rieckmann, A. M ü nscher, and C.-J. Busch. 2018. “Current Studies of Immunotherapy in Head and Neck Cancer.” Clinical Otolaryngology 43(1): 13-21.
5. Du, Xuexiang, Fei Tang, et al. 2018. “A Reappraisal of CTLA-4 Checkpoint Blockade in Cancer Immunotherapy.” Cell Research 28(4): 416-32.
6. Hirsch, Fred R et al. 2017. “Lung Cancer: Current Therapies and New Targeted Treatments.” The Lancet 389(10066): 299-311.
7. Jacobs, Joannes F M et al. 2012. “Regulatory T Cells in Melanoma: The Final Hurdle towards Effective Immunotherapy?” The Lancet Oncology 13(1): e32-42.
8. Jacobsoone-Ulrich A. et al. 2016. “Ipilimumab in anti-PD1 refractory metastatic melanoma: a report of eight cases.” Melanoma Res 26(2): 153-156.
9. Larkin J, Hodi F S, Wolchok J D. 2015. “Combined nivolumab and ipilimumab or monotherapy in untreated melanoma.” New England Journal of Medicine 373(13): 1270-1271.
10. Linsley, P. S. et al. 1994. “Human B7-1 (CD80) and B7-2 (CD86) Bind with Similar Avidities but Distinct Kinetics to CD28 and CTLA-4 Receptors.” Immunity 1(9): 793-801.
11. Motzer, R. J., McDermott, D. F., Frontera, O. Aré n et al. 2018. Nivolumab plus Ipilimumab versus Sunitinib in Advanced Renal-Cell Carcinoma. The New England Journal of Medicine 378:1277-1290
12. Ni, Ling, and Chen Dong. 2017. “New Checkpoints in Cancer Immunotherapy.” Immunological Reviews 276(1): 52-65.
13. Opdivo (nivolumab) [package insert]. Bristol-Myers Squibb Company. Princeton (NJ): 2019 [cited 2019 Apr. 30]. Available from: https://www.accessdata.fda.gov/drugsatfda_docs/label/2019/125554s070lbl.pdf
14. Pardoll, Drew M. 2012. “The Blockade of Immune Checkpoints in Cancer Immunotherapy.” Nature Reviews. Cancer 12(4): 252-64.
15. Postow, Michael A. et al. 2015. “Nivolumab and Ipilimumab versus Ipilimumab in Untreated Melanoma.” New England Journal of Medicine 372(21): 2006-17.
17. Robert C, et al. 2015. “Pembrolizumab versus ipilimumab in advanced melanoma.” New England Journal of Medicine 372(26):2521-2532.
18. Rotte, A, J Y Jin, and V Lemaire. 2018. “Mechanistic Overview of Immune Checkpoints to Support the Rational Design of Their Combinations in Cancer Immunotherapy.” Annals of Oncology 29(1): 71-83.
19. Szturz, Petr, and Jan B. Vermorken. 2017. “Immunotherapy in Head and Neck Cancer: Aiming at EXTREME Precision.” BMC Medicine 15(1): 110.
20. Tanaka, Atsushi, and Shimon Sakaguchi. 2017. “Regulatory T Cells in Cancer Immunotherapy.” Cell Research 27(1): 109-18.
21. Yau, Thomas et al. 2019. Nivolumab (NIVO)+ipilimumab (IPI) combination therapy in patients (pts) with advanced hepatocellularcarcinoma (aHCC): Results from CheckMate-040. Abstract and Poster #4012. Presented at the American Society of Clinical Oncology Annual Meeting: May 31-Jun. 4, 2019; Chicago, IL, USA
22. Yang J C, Hughes M, Kammula U, et al. Ipilimumab (Anti-CTLA4 Antibody) Causes Regression of Metastatic Renal Cell Cancer Associated With Enteritis and Hypophysitis: Journal of Immunotherapy. 2007; 30 (8):825-830.
23. Yervoy (ipilimumab) [package insert]. Bristol-Myers Squibb Company. Princeton (NJ); 2018 [cited 2019 Apr. 30]. Available from: https://www.accessdata.fda.gov/drugsatfda_docs/label/2018/125377s096lbl.pdf

1-2. (canceled)

3. A method for treating a refractory cancer in a subject, comprising administering to a subject in need thereof an anti-CTLA-4 single heavy-chain antibody, wherein the anti-CTLA-4 single heavy-chain antibody has CDR1, CDR2, and CDR3, and wherein the CDR1, the CDR2, and the CDR3 have amino acid sequences set forth in SEQ ID NOs: 1, 9, and 17, respectively.

4. The method according to claim 3, wherein the refractory cancer is not responsive or is tolerant to a PD-1/PD-L1 axis signaling pathway inhibitor.

5. The method according to claim 3, wherein the refractory cancer is primary hepatocellular carcinoma (HCC), castration-resistant prostate cancer (CRPC), renal cell carcinoma (RCC), melanoma, or colorectal cancer.

6. A method for treating cancer, comprising the step of administering to a subject in need thereof an anti-CTLA-4 single heavy-chain antibody and optionally a second therapeutic agent, wherein the second therapeutic agent is a PD-1/PD-L1 axis signaling pathway inhibitor,
wherein the anti-CTLA-4 single heavy-chain antibody has CDR1, CDR2, and CDR3, and
wherein the CDR1, the CDR2, and the CDR3 have amino acid sequences set forth in SEQ ID NOs: 1, 9, and 17, respectively.

7-9. (canceled)

10. A The method according to claim 6, wherein the anti-CTLA-4 single heavy-chain antibody is administered to the subject by intravenous drip infusion at a concentration of 0.1 mg/mL to 10.0 mg/mL, and wherein the anti-CTLA-4 single heavy-chain antibody is provided in the form of a 0.9% sodium chloride or 5% glucose solution.

11. The method according to claim 6, wherein the anti-CTLA-4 single heavy-chain antibody is administered to the subject at a dose of 0.2 mg/kg to 1 m/kg body weight.

12-19. (canceled)

20. The method according to claim 6, wherein the anti-CTLA-4 single heavy-chain antibody has a heavy chain variable region comprising the CDR1, CDR2, and CDR3, and wherein the heavy chain variable region has an amino acid sequence set forth in SEQ ID NO: 25.

21. The method according to claim 6, wherein the anti-CTLA-4 single heavy-chain antibody has an amino acid sequence set forth in SEQ ID NO: 33.

22-23. (canceled)

24. The method according to claim 6, wherein the second therapeutic agent is an anti-PD-1 monoclonal antibody.

25. The method according to claim 6, wherein the second therapeutic agent is toripalimab, perbrolizumab or nivolumab.

26. The method according to claim 6, wherein the cancer is melanoma, urothelial cancer, non-small cell lung cancer, liver cancer, neuroendocrine neoplasm or colorectal cancer.

27. The method according to claim 6, wherein the method removes a regulatory T cell (Treg) and/or increases the CD8+ T cell/Treg cell ratio in a subject.

28. The method according to claim 6, wherein the anti-CTLA-4 single heavy-chain antibody is administered to the subject by intravenous drip infusion, and the time for the intravenous drip infusion is no more than 4 hours.

29. The method according to claim 6, wherein the anti-CTLA-4 single heavy-chain antibody is administered to the subject at a dose of 0.3-0.6 mg/kg.

30. The method according to claim 6, wherein the anti-CTLA-4 single heavy-chain antibody is administered to the subject at a dose of 0.3 mg/kg to 0.6 mg/kg once every 3 weeks; the anti-CTLA-4 single heavy-chain antibody is administered to the subject at a dose of 0.3 mg/kg to 0.45 mg/kg once every 3 weeks;
wherein the anti-CTLA-4 single heavy-chain antibody is administered to the subject at a dose of 0.3 mg/kg once every week;
wherein the anti-CTLA-4 single heavy-chain antibody is administered to the subject at a dose of 0.45 mg/kg once every 3 weeks; or
wherein the anti-CTLA-4 single heavy-chain antibody is administered to the subject at a dose of 0.6 mg/kg once every 3 weeks.

31. The method according to claim 3, wherein the method removes a regulatory T cell (Treg) and/or increases the CD8+ T cell/Treg cell ratio in a subject.

32. The method according to claim 3, wherein the anti-CTLA-4 single heavy-chain antibody is administered to the subject by intravenous drip infusion at a concentration of 0.1 mg/ml to 10.0 mg/mL, and wherein the anti-CTLA-4 single heavy-chain antibody is provided in the form of a 0.9% sodium chloride or 5% glucose solution.

33. The method according to claim 3, wherein the anti-CTLA-4 single heavy-chain antibody is administered to the subject by intravenous drip infusion, and wherein the time for the intravenous drip infusion is no more than 4 hours.

34. The method according to claim 3, wherein the anti-CTLA-4 single heavy-chain antibody is administered to the subject at a dose of 0.2 mg/kg to 1 mg/kg body weight.

35. The method according to claim 3, wherein the anti-CTLA-4 single heavy-chain antibody is administered to the subject at a dose of 0.3-0.6 mg/kg.

36. The method according to claim 3, wherein the anti-CTLA-4 single heavy-chain antibody is administered to the subject at a dose of 0.3 mg/kg to 0.6 mg/kg once every 3 weeks; the anti-CTLA-4 single heavy-chain antibody is administered to the subject at a dose of 0.3 mg/kg to 0.45 mg/kg once every 3 weeks;
wherein the anti-CTLA-4 single heavy-chain antibody is administered to the subject at a dose of 0.3 mg/kg once every week;
wherein the anti-CTLA-4 single heavy-chain antibody is administered to the subject at a dose of 0.45 mg/kg once every 3 weeks; or
wherein the anti-CTLA-4 single heavy-chain antibody is administered to the subject at a dose of 0.6 mg/kg once every 3 weeks.

37. The method according to claim 3, wherein the anti-CTLA-4 single heavy-chain antibody has a heavy chain variable region comprising the CDR1, CDR2, and CDR3, and wherein the heavy chain variable region has an amino acid sequence set forth in SEQ ID NO: 25.

38. The method according to claim 3, wherein the anti-CTLA-4 single heavy-chain antibody has an amino acid sequence set forth in SEQ ID NO: 33.


SEQUENCE LISTING (40 sequences)

SEQ ID NO: 1 - protein, 10 aa, Artificial Sequence
  VH CDR1
GFTVSKNYMS

SEQ ID NO: 2 - protein, 10 aa, Artificial Sequence
  VH CDR1
GFNVSKNYMS

SEQ ID NO: 3 - protein, 10 aa, Artificial Sequence
  VH CDR1
GFNVSKNYMS

SEQ ID NO: 4 - protein, 10 aa, Artificial Sequence
  VH CDR1
GFTVSKNYMS

SEQ ID NO: 5 - protein, 10 aa, Artificial Sequence
  VH CDR1
GFTVSKNYMS

SEQ ID NO: 6 - protein, 10 aa, Artificial Sequence
  VH CDR1
GFTVSKNYMS

SEQ ID NO: 7 - protein, 10 aa, Artificial Sequence
  VH CDR1
GFTVSKNYMS

SEQ ID NO: 8 - protein, 10 aa, Artificial Sequence
  VH CDR1
GFTVSKNYMS

SEQ ID NO: 9 - protein, 16 aa, Artificial Sequence
  VH CDR2
VVYSGGSKTYADSVKG

SEQ ID NO: 10 - protein, 16 aa, Artificial Sequence
  VH CDR2
VVYSGGSKTYADSVKG

SEQ ID NO: 11 - protein, 16 aa, Artificial Sequence
  VH CDR2
VVYSGGSKTYADSVKG

SEQ ID NO: 12 - protein, 16 aa, Artificial Sequence
  VH CDR2
VVYSGGSKTYADSVKG

SEQ ID NO: 13 - protein, 16 aa, Artificial Sequence
  VH CDR2
VVYSGGSKTYADSVKG

SEQ ID NO: 14 - protein, 16 aa, Artificial Sequence
  VH CDR2
VVYSGGSKTYADSVKG

SEQ ID NO: 15 - protein, 16 aa, Artificial Sequence
  VH CDR2
VVYSGGSKTYADSVKG

SEQ ID NO: 16 - protein, 16 aa, Artificial Sequence
  VH CDR2
VVYSGGSKTYADSVKG

SEQ ID NO: 17 - protein, 11 aa, Artificial Sequence
  VH CDR3
AVPHSPSSFDI

SEQ ID NO: 18 - protein, 11 aa, Artificial Sequence
  VH CDR3
AVPHSPSSFDI

SEQ ID NO: 19 - protein, 11 aa, Artificial Sequence
  VH CDR3
AVPHSPSSFDI

SEQ ID NO: 20 - protein, 11 aa, Artificial Sequence
  VH CDR3
AVPHSPSSFDI

SEQ ID NO: 21 - protein, 11 aa, Artificial Sequence
  VH CDR3
AVPHSPSSFDI

SEQ ID NO: 22 - protein, 11 aa, Artificial Sequence
  VH CDR3
AVPHSPSSFDI

SEQ ID NO: 23 - protein, 11 aa, Artificial Sequence
  VH CDR3
AVPHSPSSFDI

SEQ ID NO: 24 - protein, 11 aa, Artificial Sequence
  VH CDR3
AVPHSPSSFDI

SEQ ID NO: 25 - protein, 119 aa, Artificial Sequence
  VH
EVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSS

SEQ ID NO: 26 - protein, 119 aa, Artificial Sequence
  VH
EVQLAETGGGLIQPGGSLRLSCAVSGFNVSKNYMSWVRQVPGKGLEWVAVVYSGGSKTYADSVKGRFILSRDNSDNTLYLQMNNLRAEDTAVYYCARAVPHSPSSFDIWGQGTMVTVSS

SEQ ID NO: 27 - protein, 119 aa, Artificial Sequence; identical to SEQ ID NO: 26
  VH

SEQ ID NO: 28 - protein, 119 aa, Artificial Sequence
  VH
EVQLAETGGGLIQPGGSLRLSCAVSGFTVSKNYMSWVRQVPGKGLEWVAVVYSGGSKTYADSVKGRFILSRDNSDNTLYLQMNNLRAEDTAVYYCARAVPHSPSSFDIWGQGTMVTVSS

SEQ ID NO: 29 - protein, 119 aa, Artificial Sequence; identical to SEQ ID NO: 25
  VH

SEQ ID NO: 30 - protein, 119 aa, Artificial Sequence; identical to SEQ ID NO: 28
  VH

SEQ ID NO: 31 - protein, 119 aa, Artificial Sequence; identical to SEQ ID NO: 25
  VH

SEQ ID NO: 32 - protein, 119 aa, Artificial Sequence; identical to SEQ ID NO: 25
  VH

SEQ ID NO: 33 - protein, 371 aa, Artificial Sequence
  heavy-chain antibody
METDTLLLWVLLLWVPGSTGEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 372 aa, Artificial Sequence
  heavy-chain antibody
METDTLLLWVLLLWVPGSTGSEVQLAETGGGLIQPGGSLRLSCAVSGFNVSKNYMSWVRQVPGKGLEWVAVVYSGGSKTYADSVKGRFILSRDNSDNTLYLQMNNLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 372 aa, Artificial Sequence
  heavy-chain antibody
METDTLLLWVLLLWVPGSTGSEVQLAETGGGLIQPGGSLRLSCAVSGFNVSKNYMSWVRQVPGKGLEWVAVVYSGGSKTYADSVKGRFILSRDNSDNTLYLQMNNLRAEDTAVYYCARAVPHSPSSFDIWGQGTMVTVSSEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 372 aa, Artificial Sequence
  heavy-chain antibody
METDTLLLWVLLLWVPGSTGSEVQLAETGGGLIQPGGSLRLSCAVSGFTVSKNYMSWVRQVPGKGLEWVAVVYSGGSKTYADSVKGRFILSRDNSDNTLYLQMNNLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 372 aa, Artificial Sequence
  heavy-chain antibody
METDTLLLWVLLLWVPGSTGSEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 372 aa, Artificial Sequence
  heavy-chain antibody
METDTLLLWVLLLWVPGSTGSEVQLAETGGGLIQPGGSLRLSCAVSGFTVSKNYMSWVRQVPGKGLEWVAVVYSGGSKTYADSVKGRFILSRDNSDNTLYLQMNNLRAEDTAVYYCARAVPHSPSSFDIWGQGTMVTVSSEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 372 aa, Artificial Sequence
  heavy-chain antibody
METDTLLLWVLLLWVPGSTGSEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 371 aa, Artificial Sequence
  heavy-chain antibody
METDTLLLWVLLLWVPGSTGEVQLVESGGGLIQPGGSLRLSCAVSGFTVSKNYMSWVRQAPGKGLEWVSVVYSGGSKTYADSVKGRFTISRDNSKNTLYLQMNSLRAEDTAVYYCARAVPHSPSSFDIWGQGTMVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK